(19) United States
(12) Patent Application Publication
(10) Pub. No.: US 2007/0259827 A1
(43) Pub. Date: Nov. 8, 2007

(54) Compositions and methods for enhancing discriminatory RNA interference

(75) Inventors: Neil Aronin, Newtonville, MA (US); Dianne Schwarz, Cambridge, MA (US); Phillip D. Zamore, Northboro, MA (US)

Correspondence Address: LAHIVE & COCKFIELD, LLP, ONE POST OFFICE SQUARE, BOSTON, MA, 02109-2127, US

(73) Assignee: UNIVERSITY OF MASSACHUSETTS, Boston, MA (US)

(21) Appl. No.: 11/698,689

(22) Filed: Jan. 25, 2007

Related U.S. Application Data
(60) Provisional application No. 60/762,225, filed on Jan. 25, 2006.
(60) Provisional application No. 60/819,707, filed on Jul. 7, 2006.

(51) Int. Cl. A61K 31/7052; A61P 25/16; A61P 25/28; C07H 21/00

(52) U.S. Cl. 514/44; 536/24.5

(57) ABSTRACT

The present invention provides methods for enhancing discriminatory RNA silencing by RNA silencing agents. In particular, the invention provides methods for generating RNA silencing agents which can discriminate between target and non-target mRNAs that differ in sequence by only one nucleotide. Also provided are improved RNA silencing agents with enhanced discriminatory RNA silencing, e.g., single nucleotide discriminatory RNA silencing. The compositions and methods of the invention are useful in therapeutic strategies for treating genetic disorders associated with dominant, gain-of-function gene mutations.

### RELATED APPLICATIONS

This application claims the benefit of U.S. Ser. No. 60/762,225, entitled “Compositions and Methods for Enhancing Discriminatory RNA Interference,” filed on Jan. 25, 2006, and U.S. Ser. No. 60/819,707, entitled “RNA Silencing Agents Capable of Single Nucleotide Discrimination,” filed on Jul. 7, 2006. The entire contents of these applications are hereby incorporated herein by reference.

### STATEMENT AS TO FEDERALLY FUNDED RESEARCH

The U.S. government may have certain rights in this invention pursuant to Grant Nos NIH 38194, R01 GM62862, R21 NS44952-01, and R01 NS38194 awarded by the National Institute of Health (NIH).

### BACKGROUND

RNA silencing refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA silencing agents which result in repression or “silencing” of a corresponding protein-coding gene. RNA silencing has been observed in many types of eukaryotes, including humans, and utility of RNA silencing agents as both therapeutics and research tools is the subject of intense interest.

Several types of small (˜19-23 nt), noncoding RNAs trigger RNA silencing in eukaryotes, including small interfering RNAs (siRNAs) and microRNAs (miRNAs, also known as small temporal RNAs (stRNAs)). Recent evidence suggests that the two classes of small RNAs are functionally interchangeable, with the choice of RNA silencing mechanism (e.g. RNAi-mediated mRNA cleavage or translational repression) determined largely by the degree of complementarity between the small RNA and its target (Schwarz and Zamore, 2002; Hutvágner and Zamore, 2002; Zeng et al., 2003; Doench et al., 2003). RNA silencing agents with a high degree of complementarity to a corresponding target mRNA have been shown to direct its silencing by the cleavage-based mechanism (Zamore et al., 2000; Elbashir et al., 2001a; Rhoades et al., 2002; Reinhart et al., 2002; Llave et al., 2002a; Llave et al., 2002b; Xie et al., 2003; Kasschau et al., 2003; Tang et al., 2003; Chen, 2003). RNA silencing agents with a lower degree of complementarity mediate gene silencing by recruiting the RISC complex to the target mRNA, thereby blocking its translation but leaving the mRNA intact (Mourelatos et al., 2002; Hutvágner and Zamore, 2002; Caudy et al., 2002; Martinez et al., 2002; Abrahante et al., 2003; Brennecke et al., 2003; Lin et al., 2003; Xu et al., 2003).

RNA silencing agents have received particular interest as research tools and therapeutic agents for their ability to knock down expression of a particular protein with a high degree of sequence specificity. The sequence specificity of RNA silencing agents is particularly useful for allele-specific silencing dominant, gain-of-function gene mutations. Diseases caused by dominant, gain-of-function gene mutations develop in heterozygotes bearing one mutant and one wild type copy of the gene. Some of the best-known diseases of this class are common neurodegenerative diseases, including Alzheimer's disease, Parkinson's disease and amyotrophic lateral sclerosis (ALS; “Lou Gehrig's disease”) (Taylor et al., 2002). In these diseases, the exact pathways whereby the mutant proteins cause cell degeneration are not clear, but the origin of the cellular toxicity is known to be the mutant protein.

One group of inherited gain-of-function disorders are characterized by one or more point mutations in an allele of a gene. For example, point mutations in the superoxide dismutase (SOD1) protein cause motor neuron degeneration that leads to ALS, because the mutant protein has acquired some toxic property (Cleveland et al., Nat. Rev. Neurosci., 2001, 2: 806-19). Neither the nature of this toxic property nor the downstream pathway that leads to the eventual motor neuron degeneration is understood. In mice, only expression of the mutant SOD1, but not elimination of SOD1 by gene knockout, causes ALS. Nonetheless, the gene knockout mice develop numerous abnormalities including reduced fertility (Matzuk et al., 1990), motor axonopathy (Shefner et al., 1999), age-associated loss of cochlear hair cells (McFadden et al., 2001) and neuromuscular junction synapses (Flood et al., 1999), and enhanced susceptibility to a variety of noxious assaults, such as excitotoxicity, ischemia, neurotoxins and irradiation, on the CNS and other systems (Matz et al., 2000; Kondo et al., 1997; Kawase et al., 1999; Behndig et al., 2001). Given the toxicity of the mutant and the functional importance of the wild-type protein, the ideal therapy for this disease would selectively block the expression of the mutant protein while retaining expression of the wild type.

Another group of inherited gain-of-function disorders are known as the trinucleotide repeat diseases. The common genetic mutation among these diseases is an increase in a series of a particular trinucleotide repeat. To date, the most frequent trinucleotide repeat is CAG, which codes for the amino acid glutamine. At least 9 CAG repeat diseases are known and there are more than 20 varieties of these diseases, including Huntington's disease, Kennedy's disease and many spinocerebellar diseases. These disorders share a neurodegenerative component in the brain and/or spinal cord. Each disease has a specific pattern of neurodegeneration in the brain and most have an autosomal dominant inheritance. The onset of the diseases generally occurs at 30 to 40 years of age, but in Huntington's disease CAG repeats in the huntingtin gene of >60 portend a juvenile onset. Research has shown that the genetic mutation (increase in length of CAG repeats from normal <36 in the huntingtin gene to >36 in disease) is associated with the synthesis of a mutant huntingtin protein, which has >36 polyglutamines (Aronin et al., 1995). It has also been shown that the protein forms cytoplasmic aggregates and nuclear inclusions (Difiglia et al., 1997) and associates with vesicles (Aronin et al., 1999). The precise pathogenic pathways are not known.

In the search for an effective treatment for these diseases, researchers in this field emphasized understanding the pathogenesis of the disease and initially sought to intercede at the level of the presumed aberrant protein interactions. However, there is no approved treatment for Huntington's disease or other trinucleotide repeat diseases. Thus, RNA silencing agents hold promise as therapy for human disease caused by increased gene activity (e.g., “gain-of-function disorders”). However, in certain instances, the sequence specificity of an RNA silencing agent may be sub-optimal, particularly where the desired target mRNA differs in sequence from an essential, non-target mRNA (e.g., an mRNA encoded by a wild-type allele) by only a few nucleotides (e.g., an mRNA by a single point mutation or single nucleotide polymorphism or “SNP”). Although RNA silencing agents can discriminate among alleles that differ in sequence by a single nucleotide, they can occasionally retain a capacity for a low (but nonetheless undesirable) level of non-target allele silencing. This may be particularly problematic for the treatment of gain-of-function disorders where silencing of the gain-of-function allele is desired, but expression of the wild-type allele must be maintained due to the essential function that it serves in the organism. Accordingly, improved RNA silencing agents capable of enhanced discriminatory RNA, and single nucleotide discrimination in particular, are urgently needed.

### SUMMARY

The invention is based, at least in part, on the discovery that the positioning of a specificity-determining nucleotide within an RNA silencing agent is critical to ensure reliable discriminatory RNA silencing activity by the agent. In particular, placement of a specificity-determining nucleotide in the central or 3′ regions of an RNA silencing agent (e.g. an siRNA) ensures single-nucleotide discrimination between a target, mutant mRNA to which the specificity determining nucleotide is complementary, and a non-target, wild-type mRNA with which the specificity determining nucleotide forms a mismatched or wobble base pair. Surprisingly, positioning the specificity-determining nucleotide in the 5′ end of the siRNA (also known as the “seed region” of the siRNA) does not ensure good single-nucleotide discrimination, despite the importance of this region in determining the selectivity of target binding. Accordingly, RNA silencing agents synthesized according to the methods the invention have improved allelic discrimination and facilitate the silencing of a harmful gene product, while preserving the ability of the normal or wild-type mRNA to fulfill its function.

The invention is also based on the discovery that substitution of one or more nucleotides of an RNA silencing agent with destabilizing nucleotides can also improve discriminatory RNA silencing activity by the RNA silencing agent. In particular, substitution of nucleotides in the antisense strand of the RNA silencing agent can diminish or abolish the ability of the agent to direct RNA silencing against non-target mRNAs (e.g. non-target, wild-type mRNAs having a single nucleotide mismatch with the antisense strand of the RNA silencing agent). In addition, the ability of the modified RNA silencing agent to mediate RNA silencing of a target mRNA (e.g. a mutant mRNA containing a single nucleotide polymorphism) is maintained.

In certain aspects, the invention is directed to RNA silencing agents capable of enhanced discriminatory RNA silencing wherein a specificity-determining nucleotide of the RNA silencing agent is positioned within the central or the 3′ end of the antisense strand of said agent. The specificity-determining nucleotide within the antisense strand of the RNA silencing agent forms mismatch or wobble base pair with the non-target RNA. In other aspects, the invention is directed to methods of enhancing discriminatory RNA silencing of an RNA silencing agent comprising positioning a specificity-determining nucleotide within the central or 3′ ends of the antisense strand of said agent such that the specificity determining nucleotide forms a mismatched or wobble base pair with a non-target RNA (e.g. RNA corresponding to a wild-type allele of a gain-of-function protein or a non-target SNP allele).

In one aspect, the invention provides a RNA silencing agent capable of enhanced discriminatory RNA silencing wherein the RNA silencing agent comprises at least one specificity determining nucleotide within the central or the 3′ end of the antisense strand of said agent, wherein the specificity-determining nucleotide forms a mismatch or wobble base pair between the antisense strand of the RNA silencing agent and a non-target RNA. In certain embodiments, the RNA silencing agent is a siRNA.

In other embodiments, the specificity-determining nucleotide is located or positioned at a nucleotide position selected from the group consisting of P8, P9, P10, P12, P13, P14, P15, P16 and 19, wherein the nucleotide position is relative to the 5′end of the antisense strand. In yet other embodiments, the specificity-determining nucleotide is located at a nucleotide position selected from the group consisting of P9, P10, P12, P13, P14, and P16, wherein the nucleotide position is relative to the 5′end of the antisense strand.

In another embodiment, the specificity determining nucleotide is located at nucleotide position 10 relative to the 5′ end of the antisense strand. In another embodiment, the specificity determining nucleotide is located at nucleotide position 16 relative to the 5′ end of the antisense strand.

In another embodiment, the non-target RNA is encoded by a wild-type allele corresponding to the mutant allele of a gene encoding a mutant gain-of-function protein. In one embodiment, said mutant gain-of-function protein is a mutant SOD1 protein. In another embodiment, said mutant gain-of-function protein is a mutant Huntingtin protein.

In other embodiments, the specificity determining nucleotide is complementary to the target mRNA.

In other embodiments, the specificity determining nucleotide forms a purine:purine mismatch with the non-target mRNA. In one embodiment, the purine:purine mismatch is a G:G mismatch. In another embodiment, the purine:purine mismatch is a G:A mismatch.

In other embodiments, the RNA silencing agent provides more than 4-fold discrimination between two alleles which differ by at least one nucleotide. In yet other embodiments, the RNA silencing agent provides more than 20-fold discrimination between two alleles which differ by at least one nucleotide.

In yet other aspects, the invention is directed to methods of enhancing discriminatory RNA silencing by a RNA silencing agent comprising substituting at least one nucleotide within an antisense strand of said agent with a destabilizing nucleotide, such that discriminatory RNA silencing by said RNA silencing agent is enhanced. In other aspects, the invention is directed to RNA silencing capable of enhanced discriminatory RNA silencing.

In one embodiment, the RNA silencing agents of the invention comprise an antisense strand having a G/C content of greater than 40%. In other embodiments, the RNA silencing agent is capable of inducing the discriminatory RNA silencing of a target sequence having a G/C content of greater than 40%.

In certain embodiments, the melting temperature (Tm) of a duplex formed by the antisense strand of the RNA silencing agent and the corresponding target mRNA sequence is decreased. In exemplary embodiments, the Tm is decreased by more than 0.5° C. In a particular embodiment, the Tm is decreased by less than 2° C.

In certain embodiments, the destabilizing nucleotide is a universal base. In preferred embodiments, the universal base is selected from the group consisting of inosine and 2′-deoxyinosine. In other embodiments, the destabilizing nucleotide is capable of forming a mismatched base pair or a wobble base pair. In yet other embodiments, the destabilizing nucleotide is capable of forming an ambiguous base pair.

In certain embodiments, the RNA silencing agents of the invention comprise a specificity-determining nucleotide within the antisense strand that is a G or C. In other embodiments, the RNA silencing agent is capable of single nucleotide discrimination.

In other certain embodiments, the RNA silencing agents of the invention comprise an antisense strand that complementary to a mutant allele such that the RNA silencing agent is capable of substantially silencing the mutant allele without substantially silencing the corresponding wild-type allele. In other embodiments, the target sequence of the RNA silencing agent is a polymorphic target sequence comprising 1-3 contiguous nucleotides. In yet other embodiments, the G/C content of the sequence formed by nucleotide positions 1-5 on the 5′ side and positions 1-5 on the 3′ side of the polymorphic target sequence is greater than 50%.

In certain embodiments, the RNA silencing agents of the invention comprise a destabilizing nucleotide that is present at a position within 5 nucleotides of a specificity-determining nucleotide. In particular embodiments, the destabilizing nucleotide is present at a position that is 3 nucleotides from the specificity-determining nucleotide. In other embodiments, the destabilizing nucleotide forms a base pair with a cytosine in the target sequence.

In certain embodiments, the target sequence of the RNA silencing agents of the invention is a polymorphic target sequence, for example a sequence comprising a single nucleotide polymorphism. In particular embodiments, the single nucleotide polymorphism is a guanine or cytosine. In other embodiments, the polymorphic target sequence is a point mutation. In a particular embodiment, the point mutation is a guanine or cytosine. In yet other embodiments, the polymorphic sequence is correlated with a disorder associated with a dominant gain of function mutation.

In certain embodiments, the sequence of the antisense strand of the RNA silencing agent and its target sequence differ by seven or fewer base pairs out of 21 contiguous base pairs. In other embodiments, the sequence of the antisense strand and the target sequence differ by one base pair out of 21 base pairs.

In certain embodiments, the antisense strand of the RNA silencing agent is capable of forming a stable duplex with the target mRNA under physiological conditions.

In other embodiments, the RNA silencing agent is capable of (i) substantially silencing the target sequence, and (ii) not substantially silencing the non-target sequence at a concentration greater than 0.05 nM. In a particular embodiment, the RNA silencing agent is capable of (i) substantially silencing the target sequence, and (ii) not substantially silencing the non-target sequence at a concentration up to 50 nM.

In an exemplary embodiment, the RNA silencing agent is a siRNA. In other exemplary embodiments, the RNA silencing agent is a shRNA.

Expression cassettes encoding an RNA silencing agent of the invention and vectors comprising said cassettes are also contemplated by the invention. In certain aspects, the invention is also directed to host cells comprising said vectors or said expression cassettes.

In other aspects, the invention is directed to therapeutic compositions comprising a RNA silencing agent of the invention, together with a pharmaceutically acceptable carrier.

In yet other aspects, the invention is directed to a method of conducting discriminatory RNA interference (RNAi) in a cell comprising a first allelic sequence having an allelic polymorphism relative to a second allelic sequence, the method comprising contacting the cell with an RNA silencing agent of the invention, such that the first allelic sequence is selectively targeted.

In still further aspects, the invention is directed to a method of substantially silencing a targeted allele in a cell while allowing substantially continued expression of a wild-type allele in the cell comprising contacting the cell with an RNA silencing agent of the invention, such that expression from the targeted allele is substantially silenced while expression of the wild-type allele is not substantially silenced.

In other aspects, the invention is directed to a method of treating a subject having a disease or disorder correlated with the presence of a dominant gain of function mutant allele, the method comprising administering to the subject a therapeutically effective amount of an RNA silencing agent of the invention.

In still other aspects, the invention is directed to a method of treating a subject having or at risk for a disease or disorder characterized or caused by a gain-of-function mutant protein, comprising: administering to said subject an effective amount of a RNA silencing agent of the invention and thereby targeting an allelic polymorphism within a gene encoding said mutant protein, such that sequence-specific RNA silencing of said gene occurs; thereby treating said disease in said subject. In certain embodiments, the disease is a neurodegenerative disease. In an exemplary embodiment, the neurodegenerative disease is selected from the group of amyotrophic lateral sclerosis, Huntington's disease, Alzheimer's disease, Parkinson's disease, and spinocerebellar ataxia (SCA). In another particular embodiment, the neurodegenerative disease is a trinucleotide-repeat disease (e.g., a disease associated with polyglutamine repeats). In an exemplary embodiment, the trinucleotide repeat disease is a disease selected from the group consisting of Huntington's disease, spino-cerebellar ataxia type 1, spino-cerebellar ataxia type 2, spino-cerebellar ataxia type 3, spino-cerebellar ataxia type 6, spino-cerebellar ataxia type 7, spino-cerebellar ataxia type 8, spino-cerebellar ataxia type 12, fragile X syndrome, fragile XE MR, Friedreich ataxia, myotonic dystrophy, spinal bulbar muscular disease and dentatoiubral-pallidoluysian atrophy.

In a particular embodiment, the invention is directed to a method of treating amyotrophic lateral sclerosis using an RNA silencing agent of the invention directed to a gain-of-function allele associated with said disease (e.g. SOD1). In an exemplary embodiment, the gain-of-function allele comprises a mutation selected from the group consisting of G256C and G281C.

In other aspects, the invention is directed to methods of screening for RNA silencing agents having discriminatory RNAi comprising (a) contacting a cell containing a predetermined mutant allele with an RNA silencing agent of the invention comprising an antisense strand with complementarity to the mutant allele, wherein said antisense strand further comprises a destabilizing base or analog thereof; b) contacting a cell containing a wild-type allele with said RNA silencing agent, and (c) determining if the mutant allele is substantially silenced while the wild-type allele retains substantially normal activity.

### BRIEF DESCRIPTION OF THE DRAWINGS

FIG. 1 depicts guide strand sequences (SEQ ID NOs:1-19) of a tiled set of siRNAs (P1-P19) which target the G85R point mutation in human SOD1 mRNA. Each guide stand sequence is perfectly complementary to an mRNA sequence corresponding to the point mutation in the mutant allele of SOD1 (SEQ ID NO:20) while forming a G:G mismatch with the non-target, wild-type SOD1 mRNA sequence (SEQ ID NO:21). The position of the nucleotide forming the mismatch is indicated by grey shading. The third nucleotide from the 3′ end of the sense strand (not depicted) of each siRNA contains a mismatch with the guide sequence, thereby creating an functionally asymmetric siRNA having an unpaired 5′ end which facilates entry of the guide strand into RISC.

FIG. 2 depicts the results of an analysis of the rate of mRNA cleavage in Drosophila embryo lysate over a 2 hour period for each siRNA depicted in FIG. 1. Cleavage of mutant SOD1 mRNA is represented by filled circles, while cleavage of wild-type SOD1 mRNA is represented by open circles.

FIG. 3 depicts the results of an analysis of the rate of mRNA cleavage in Drosophila embryo lysate over a 2 hour period by a fully 5′-paired P11 siRNA. Annotation is the same as for FIG. 2.

FIG. 4 depicts the results of an analysis of the initial rate of mRNA cleavage in Drosophila embryo lysate under single-turnover reaction conditions for each siRNA depicted in FIG. 1. Annotation is the same as for FIG. 2.

FIG. 5 depicts the results of an analysis of the rate of non-target, wild-type mRNA cleavage in Drosophila embryo lysate over a 24 hour incubation period by siRNAs P5, P9, P10, P12-16, and P19.

FIG. 6 depicts the results of an analysis of siRNA-mediated mRNA cleavage in cultured human HEK 293T cells transfected with 2 nM (grey bars) or 20 nM (white bars) of a reporter plasmid containing a fusion of the sequence encoding Photinus pyralis (Pp) firefly luciferase with a sequence comprising either (i) the target, mutant G85R allele of SOD1 (FIG. 6A), (ii) the wild-type (mismatched) allele of SOD1 (FIG. 6B), or (iii) a sequence comprising a U at position 323 of the wild-type allele of SOD1 (FIG. 6C). Cleavage is depicted on the y-axis in terms of relative expression of Pp luciferase and an untargeted Renilla (Rr) luciferase control.

FIG. 7 shows a heat map depicting the results of a microarray analysis of gene expression in cultured HeLa cells transfected with the SOD1 siRNAs depicted in FIG. 1. “Off-target” regulated genes are shown in columns, while experiments with each siRNA are shown in rows. The scale bar indicates log₁₀ expression ratio of transfected/mock transfected cells. Genes having decreased mRNA levels compared to mock transfected cells are indicated by light grey or white shading (ie. log(ratio) of approximately −0.6), while genes having increased mRNA levels are indicated by dark grey shading (ie. log(ratio of approximately +0.6). Unregulated genes are indicated by black shading (ie. log(ratio of approximately 0). The arrow indicates the position of the wild-type SOD1 mRNA, compared to a mock HeLa cell transfection.

FIG. 8 depicts the results of a quantitative RT-PCR analysis of endogenous SOD1 mRNA levels following siRNA transfection of cultured human HeLa cells. The data depicted is the mean±standard deviation of three replicate determinations for each siRNA. The height of each bar is correlated with allele discrimination. In particular, taller bars imply greater discrimination against the mismatched target RNA.

FIG. 9A depicts the silencing activity of different concentrations of a fully base-paired p10 siRNA against a firefly luciferase reporter containing sense (open squares) or antisense (filled squares) target SOD1 sequences. FIG. 9B depicts the discriminatory silencing activity of P10 siRNAs forming different types of single nucleotide siRNA:mRNA base pairs between position 10 of the guide strand of the siRNA and a target mRNA. FIG. 9C depicts the dose-dependent discriminatory silencing activity of p10 siRNA forming pyrimidine mismatches (U:C (triangles), U:U (diamonds), U:G (circles)) or a perfect U:A match (squares) with the point mutation in a target mRNA. FIG. 9D depicts dose-dependent discriminatory silencing activity of p10 siRNA forming purine:purine mismatches (A:G (circles); A:A (squares); A:C (triangles)) or an A:U match with the point mutation in a target mRNA.

FIG. 10 depicts the results of an analysis of the effect of purine:purine (siRNA:mRNA) mismatches on discriminatory silencing activity of a P10 siRNA. Mismatches were introduced by substitution with purine residues at nucleotide positions N1-N19 along the guide strand of a P10 siRNA. Taller bars correspond to greater single nucleotide discrimination.

FIGS. 11A-F depict discriminatory RNAi mediated by unmodified siRNAs (FIGS. 11A-C) and Inosine-modified siRNAs (FIGS. 11D-F). (A) Graph depicting the potency of an unmodified siRNA in silencing the expression of a fully complementary target huntingtin mRNA by RNA interference. (B) Graph depicting the residual silencing of a non-target huntingtin mRNA by the unmodified siRNA duplex. (C) Schematic showing the sense (SEQ ID NO:32) and antisense (SEQ ID NO:33) strands of the unmodified siRNA and the relevant sequence portions of both the target huntingtin mRNA sequence (SEQ ID NO:34) corresponding to a mutant gain-of-function allele having a single nucleotide polymorphism and a non-target huntingtin mRNA sequence (SEQ ID NO:35) corresponding to a wild-type allele. The antisense-strand of the siRNA is fully complementary (“matched”) to the target huntingtin mRNA, but contains a mismatch (G:G) with the wild-type huntingtin RNA. The position of the single nucleotide polymorphism and the corresponding wild-type nucleotide is indicated by capitalization. (D) Graph depicting the effectiveness of the Inosine-modified siRNA in mediating RNAi of the fully complementary target huntingtin mRNA. (E) Graph depicting the inability of the Inosine-modified siRNA to mediate RNAi of the non-target huntingtin mRNA sequence. (F) Schematic showing the position of inosine residues within the antisense (AS) strand of the Inosine-modified siRNA (SEQ ID NO:36).

### DETAILED DESCRIPTION

The present invention is based on the discovery that RNA silencing agents (e.g., siRNA and shRNA) can selectively inhibit the expression of a mutant allele, even when the mutant mRNA differs from wild-type by only a single nucleotide, as is the case with certain mutations, e.g., mutations of SOD1 correlated with ALS. These methods are applicable to the treatment of diseases that are caused by dominant, gain-of-function type of gene mutations, including, but not limited to, ALS. The RNA silencing agents (e.g. siRNAs) of the present invention are capable of single nucleotide discrimination and selectively down-regulating expression of their target genes.

In certain aspects, the invention relates to methods and reagents for treating or preventing a variety of diseases characterized by a mutation (e.g., a point mutation that leads to a gain-of-function) in one allele or copy of a gene, the mutation encoding a protein which is sufficient to contribute to or cause the disease. Preferably, the methods and reagents are used to treat diseases caused or characterized by a mutation that is inherited in an autosomal dominant fashion.

In other aspects, the invention relates to methods and reagents for treating or preventing diseases characterized by mutations or allelic polymorphisms (e.g., single nucleotide polymorphisms or “SNPs”) that do not themselves cause a disease phenotype but are linked with a disease allele, and not its wild-type counterpart. By targeting the SNP isoform present in the disease allele, expression of the disease-causing allele might by selectively reduced without altering expression of the wild-type allele.

The methods of the invention utilize RNA silencing technology (e.g. RNAi) against selected mutations (e.g., point mutations) or allelic polymorphisms (e.g., SNPs) occurring in, or associated with, a single allele in the mutant gene encoding a gain-of-function mutant protein, e.g., the point mutation in the copper zinc superoxide dismutase (SOD1) gene associated with amyotrophic lateral sclerosis (ALS). RNA silencing destroys the corresponding mutant mRNA with single nucleotide specificity and selectivity. RNA silencing agents of the present invention are targeted to polymorphic regions of a mutant gene, resulting in cleavage or translational repression of mutant mRNA. These RNA silencing agents, through a series of protein-nucleotide interactions, function to cleave or translationally repress the mutant mRNAs.

So that the invention maybe more readily understood, certain terms are first defined:

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence”, e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g. promoter or enhancer elements) of a target gene. In other embodiments, the target polynucleotide sequence is a target mRNA encoded by a target gene.

As used herein, the term “target gene” is a gene whose expression is to be substantially inhibited or “silenced.” This silencing can be achieved by RNA silencing, e.g. by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms. In another embodiment, the target and non-target genes can share less than 100% sequence identity. In another embodiment, the non-target gene may be a homolog (e.g. an ortholog or paralog) of the target gene.

A “target allele” is an allele whose expression is to be selectively inhibited or “silenced.” This silencing can be achieved by RNA silencing, e.g. by cleaving the mRNA of the target gene or target allele by a siRNA. The term “non-target allele” is an allele whose expression is not to be substantially silenced. In certain embodiments, the target and non-target alleles can correspond to the same target gene. In other embodiments, the target allele corresponds to a target gene, and the non-target allele corresponds to a non-target gene. In one embodiment, the polynucleotide sequences of the target and non-target alleles can differ by one or more nucleotides. In another embodiment, the target and non-target alleles can differ by one or more allelic polymorphisms. In another embodiment, the target and non-target alleles can share less than 100% sequence identity.

The term “polymorphism” as used herein, refers to a variation (e.g., one or more deletions, insertions, or substitutions) in a gene sequence that is identified or detected when the same gene sequence from different sources or subjects (but from the same organism) are compared. For example, a polymorphism can be identified when the same gene sequence from different subjects are compared. Identification of such polymorphisms is routine in the art, the methodologies being similar to those used to detect, for example, breast cancer point mutations. Identification can be made, for example, from DNA extracted from a subject's lymphocytes, followed by amplification of polymorphic regions using specific primers to said polymorphic region. Alternatively, the polymorphism can be identified when two alleles of the same gene are compared.

A variation in sequence between two alleles of the same gene within an organism is referred to herein as an “allelic polymorphism”. The polymorphism can be at a nucleotide within a coding region but, due to the degeneracy of the genetic code, no change in amino acid sequence is encoded. Alternatively, polymorphic sequences can encode a different amino acid at a particular position, but the change in the amino acid does not affect protein function. Polymorphic regions can also be found in non-encoding regions of the gene.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, and dual-function oligonucleotides as well as precursors thereof. In one embodiment, the RNA silencing agent is capable of inducing RNA interference. In another embodiment, the RNA silencing agent is capable of mediating translational repression.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, ²N-methylguanosine and ^{2,2}N,N-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, ²N-methylguanosine and ^{2,2}N,N-dimethylguanosine.

The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of preferred modified nucleotides include, but are not limited to, 2-amino-guanosine, 2-amino-adenosine, 2,6-diamino-guanosine and 2,6-diamino-adenosine. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O— and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., August 2000 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. April 2000 10(2):117-21, Rusckowski et al. Antisense Nucleic Acid Drug Dev. October 2000 10(5):333-45, Stein, Antisense Nucleic Acid Drug Dev. October 2001 11(5): 317-25, Vorobjev et al. Antisense Nucleic Acid Drug Dev. April 2001 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. The oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, and/or phosphorothioate linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA silencing (e.g. RNA interference). In an exemplary embodiment, oligonucleotides comprise Locked Nucleic Acids (LNAs) or Peptide Nucleic Acids (PNAs).

As used herein the term “destabilizing nucleotide” refers to a first nucleotide or nucleotide analog capable of forming a base pair with second nucleotide or nucleotide analog such that the base pair is of lower bond strength than a conventional base pair (ie. Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, Van der Waals interactions, and the like between said nucleotides (or nucleotide analogs).

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, Van der Waals interactions, and the like between said nucleotides (or nucleotide analogs).

As used herein, the term “mismatched base pair” refers to a base pair consisting of noncomplementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g. double-stranded DNA or RNA) due to stacking interactions. The base portion of a universal nucleotide typically comprises a nitrogen-containing aromatic heterocyclic moiety.

As used herein, the term “specificity-determining nucleotide” refers to a nucleotide or base which is capable of discriminating between bases or nucleotides on a target nucleic acid. In particular, the term “specificity-determining nucleotide” refers to a nucleotide within an RNA silencing agent that forms a complementary base pair (e.g., a Watson-Crick base pair) with a polymorphic residue in a target nucleic acid. For example, in a RNA silencing agent that is complementary to a target mRNA sequence having a single nucleotide polymorphism (SNP), the specificity-determining nucleotide forms a complementary base pair with the SNP. In other particular embodiments, the specificity-determining nucleotide also forms a mismatched or wobble base pair with the corresponding non-polymorphic residue in a non-target nucleic acid. As used herein, the phrase “specificity-determining position of a RNA silencing agent” means the location of the specificity-determining nucleotide in a strand of the RNA silencing agent. As used herein, the phrase “specificity-determining position of a target mRNA”, means the location of the nucleotide that is complementary to the specificity-determining nucleotide when the RNA silencing agent is aligned with said target mRNA sequence.

As used here, the term “melting temperature” or “Tm” refers to the temperature at which approximately 50% of a population of double-stranded polynucleotide molecules becomes dissociated into single strands.

As used herein, the terms “sufficient complementarity” or “sufficient degree of complementarity” mean that the RNA silencing agent has a sequence (e.g. in the antisense strand, mRNA targeting moiety or miRNA recruiting moiety) which is sufficient to bind the desired target RNA, respectively, and to trigger the RNA silencing of the target mRNA.

As used herein, the term “asymmetry”, as in the asymmetry of the duplex region of an RNA silencing agent (e.g. the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g, single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the “5′ end”, as in the 5′ end of an antisense strand, refers to the 5′ terminal nucleotides, e.g., between one and about 5 nucleotides at the 5′ terminus of the antisense strand. As used herein, the “3′ end”, as in the 3′ end of a sense strand, refers to the region, e.g., a region of between one and about 5 nucleotides, that is complementary to the nucleotides of the 5′ end of the complementary antisense strand.

As used herein, the term “RNA interference” (“RNAi”) refers to a type of RNA silencing which results in the selective intracellular degradation of a target RNA. RNAi occurs in cells naturally to remove foreign RNAs (e.g., viral RNAs). Natural RNAi proceeds via fragments cleaved from free dsRNA which direct the degradative mechanism to other similar RNA sequences. Both RNAi and translational repression are mediated by RISC. Both RNAi and translational repression occur naturally or can be initiated by the hand of man, for example, to silence the expression of target genes.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

An RNA silencing agent having a strand which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. Preferably, a siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising ˜21 nucleotides (or nucleotide analogs), for example, 19, 20, 21 or 22 nucleotides. The term “long” siRNA refers to a siRNA comprising ˜24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “microRNA” (“miRNA”), also referred to in the art as “small temporal RNAs” (“stRNAs”), refers to a small (10-50 nucleotide) RNA which are genetically encoded (e.g. by viral, mammalian, or plant genomes) and are capable of directing or mediating RNA silencing. A “miRNA disorder” shall refer to a disease or disorder characterized by an aberrant expression or activity of a miRNA.

As used herein, the term “dual functional oligonucleotide” refers to a RNA silencing agent having the formula T-L-μ, wherein T is an mRNA targeting moiety, L is a linking moiety, and μ is a miRNA recruiting moiety. As used herein, the terms “mRNA targeting moiety”, “targeting moiety”, “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA). As used herein, the term “linking moiety” or “linking portion” refers to a domain, portion or region of the RNA-silencing agent which covalently joins or links the mRNA.

As used herein, the term “antisense strand” of an RNA silencing agent, e.g. an siRNA or RNAi agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g. a siRNA or RNAi agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by man. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

An “isolated nucleic acid molecule or sequence” is a nucleic acid molecule or sequence that is not immediately contiguous with both of the coding sequences with which it is immediately contiguous (one on the 5′ end and one on the 3′ end) in the naturally occurring genome of the organism from which it is derived. The term therefore includes, for example, a recombinant DNA or RNA that is incorporated into a vector; into an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide sequence.

As used herein, the term “isolated RNA” (e.g., “isolated shRNA”, “isolated siRNA”, “isolated siRNA-like duplex”, “isolated miRNA”, “isolated gene silencing agent”, or “isolated RNAi agent”) refers to RNA molecules which are substantially free of other cellular material, or culture medium when produced by recombinant techniques, or substantially free of chemical precursors or other chemicals when chemically synthesized.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

A gene “involved” in a disease or disorder includes a gene, the normal or aberrant expression or function of which effects or causes the disease or disorder or at least one symptom of said disease or disorder.

“Allele specific inhibition of expression” refers to the ability to significantly inhibit expression of one allele of a gene over another, e.g., when both alleles are present in the same cell. For example, the alleles can differ by one, two, three or more nucleotides. In some cases, one allele is associated with disease causation, e.g., a disease correlated to a dominant gain-of-function mutation.

The term “gain-of-function mutation” as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene which gives rise to the change in the function of the encoded protein. In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein.

The term “polyglutamine domain,” as used herein, refers to a segment or domain of a protein that consist of a consecutive glutamine residues linked to peptide bonds. In one embodiment the consecutive region includes at least 5 glutamine residues.

The term “expanded polyglutamine domain” or “expanded polyglutamine segment”, as used herein, refers to a segment or domain of a protein that includes at least 35 consecutive glutamine residues linked by peptide bonds. Such expanded segments are found in subjects afflicted with a polyglutamine disorder, as described herein, whether or not the subject has shown to manifest symptoms.

The term “trinucleotide repeat” or “trinucleotide repeat region” as used herein, refers to a segment of a nucleic acid sequence e.g.,) that consists of consecutive repeats of a particular trinucleotide sequence. In one embodiment, the trinucleotide repeat includes at least 5 consecutive trinucleotide sequences. Exemplary trinucleotide sequences include, but are not limited to, CAG, CGG, GCC, GAA, CTG, and/or CGG.

The term “trinucleotide repeat diseases” as used herein, refers to any disease or disorder characterized by an expanded trinucleotide repeat region located within a gene, the expanded trinucleotide repeat region being causative of the disease or disorder. Examples of trinucleotide repeat diseases include, but are not limited to spino-cerebellar ataxia type 12 spino-cerebellar ataxia type 8, fragile X syndrome, fragile XE Mental Retardation, Friedreich's ataxia and myotonic dystrophy. Preferred trinucleotide repeat diseases for treatment according to the present invention are those characterized or caused by an expanded trinucleotide repeat region at the 5′ end of the coding region of a gene, the gene encoding a mutant protein which causes or is causative of the disease or disorder. Certain trinucleotide diseases, for example, fragile X syndrome, where the mutation is not associated with a coding region may not be suitable for treatment according to the methodologies of the present invention, as there is no suitable mRNA to be targeted by RNAi. By contrast, disease such as Friedreich's ataxia may be suitable for treatment according to the methodologies of the invention because, although the causative mutation is not within a coding region (i.e., lies within an intron), the mutation may be within, for example, an mRNA precursor (e.g., a pre-spliced mRNA precursor).

The term “polyglutamine disorder” as used herein, refers to any disease or disorder characterized by an expanded of a (CAG)ₙ repeats at the 5′ end of the coding region (thus encoding an expanded polyglutamine region in the encoded protein). In one embodiment, polyglutamine disorders are characterized by a progressive degeneration of nerve cells. Examples of polyglutamine disorders include but are not limited to: Huntington's disease, spino-cerebellar ataxia type 1, spino-cerebellar ataxia type 2, spino-cerebellar ataxia type 3 (also know as Machado-Joseph disease), and spino-cerebellar ataxia type 6, spino-cerebellar ataxia type 7 and dentatoiubral-pallidoluysian atrophy.

The phrase “examining the function of a gene in a cell or organism” refers to examining or studying the expression, activity, function or phenotype arising therefrom.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

“Treatment”, or “treating” as used herein, is defined as the application or administration of a therapeutic agent (e.g., a RNA silencing agent or a vector or transgene encoding same) to a patient, or application or administration of a therapeutic agent to an isolated tissue or cell line from a patient, who has a disorder with the purpose to cure, heal, alleviate, relieve, alter, remedy, ameliorate, improve or affect the disease or disorder, or symptoms of the disease or disorder. The term “treatment” or “treating” is also used herein in the context of administering agents prophylactically. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. The term “patient” includes human and other mammalian subjects that receive either prophylactic or therapeutic treatment.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Various aspects of the invention are described in further detail in the following subsections.

I. RNA Silencing Agents

The present invention features improved RNA silencing agents (e.g., siRNA and shRNAs), methods of making said improved RNA silencing agents, and methods (e.g., research and/or therapeutic methods) for using said improved RNA silencing agents (or portions thereof) for discriminatory RNA silencing. The RNA silencing agents of the invention are duplex molecules (or molecules having duplex-like structure) comprising a sense strand and a complementary antisense strand (or portions thereof), wherein the antisense strand has sufficient complementary to a target sequence (e.g. target mRNA) to mediate an RNA-mediated silencing mechanism (e.g. RNAi) with enhanced discrimination. In certain embodiments, the target sequence may be an allelic polymorphism or point mutation which is unique to a mutant allele for which silencing is desired.

a) Design of Conventional siRNA Molecules

An siRNA molecule of the invention is a duplex consisting of a sense strand and complementary antisense strand, the antisense strand having sufficient complementary to a target mRNA sequence to direct a target-specific RNA silencing mechanism. In preferred embodiments, the antisense strand has sufficient complementary to the target mRNA to direct RNA interference (RNAi), as defined herein, i.e., the siRNA has a sequence sufficient to trigger the destruction of the target mRNA by the RNAi machinery or process.

Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target region e.g., a gain-of-function gene target region, and the other strand is identical or substantially identical to the first strand.

Generally, siRNAs can be designed by using any method known in the art, for instance, by using the following protocol:

1. A target mRNA is selected (e.g., a mutant allele or mRNA) having target sequence or region with at least one mismatch (e.g., a single nucleotide mismatch) as compared to a reference or non-target mRNA sequence (e.g., a wild type allele or mRNA sequence). In other words, the target mRNA differs by at least one nucleotide from the non-target mRNA within the targeted region. In one embodiment, the mismatch may be a point mutation (e.g. a gain-of-function point mutation e.g. a SOD1 point mutation). In one embodiment, the target region comprises a gain of function point mutation within a protein coding region. In another embodiment, the mismatch is a polymorphism (e.g a polymorphism outside a coding region of the target gene). Exemplary polymorphisms are selected from the 5′ untranslated region of a target gene. Cleavage of mRNA at these sites should eliminate translation of corresponding mutant protein. Polymorphisms from other regions of the mutant gene are also suitable for targeting. In another embodiment, the target region comprises a portion of the target gene (e.g., the htt gene) that includes the.

In preferred embodiments, the target sequence or region further comprises AA dinucleotide sequences; each AA and the 3′ adjacent 16 or more nucleotides are potential siRNA targets. In another preferred embodiment, the nucleic acid molecules are selected from a region of the target allele sequence beginning at least 50 to 100 nt downstream of the start codon, e.g., of the sequence of the target mRNA. Further, siRNAs with lower G/C content (35-55%) may be more active than those with G/C content higher than 55%. Thus in one embodiment, the invention includes target sequences having 35-55% G/C content, although the invention is not limited in this respect.

2. The siRNA should be specific for the target region of the target mRNA. Accordingly, the sense strand of the siRNA is designed based on the sequence of the selected target site. Preferably the sense strand includes about 19 to 25 nucleotides, e.g., 19, 20, 21, 22, 23, 24 or 25 nucleotides. More preferably, the sense strand includes 21, 22 or 23 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant invention provided that they retain the ability to mediate RNAi. Longer RNA silencing agents have been demonstrated to elicit an interferon or PKR response in certain mammalian cells which may be undesirable. Preferably the RNA silencing agents of the invention do not elicit a PKR response (i.e., are of a sufficiently short length). However, longer RNAi agents may be useful, for example, in cell types incapable of generating a PRK response or in situations where the PKR response has been downregulated or dampened by alternative means.

The siRNA molecules of the invention have sufficient complementarity with the target site such that the siRNA can mediate RNAi. In general, siRNA containing nucleotide sequences sufficiently identical to a portion of the target gene to effect RISC-mediated cleavage of the target gene are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99%, or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between the wild type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The sense strand sequence is designed such that the mutation or polymorphism is essentially in the middle of the strand. For example, if a 21-nucleotide siRNA is chosen, the polymorphism is at, for example, nucleotide 6, 7, 8, 9, 10, 11, 12, 13, 14, 15 or 16 (i.e., 6, 7, 8, 9, 10, 11, 12, 13, 14, 15 or 16 nucleotides from the 5′ end of the sense strand. For a 22-nucleotide siRNA, the polymorphism is at, for example, nucleotide 7, 8, 9, 10, 11, 12, 13, 14, 15 or 16. For a 23-nucleotide siRNA, the polymorphism is at, for example, 7, 8, 9, 10, 11, 12, 13, 14, 15 or 16. For a 24-nucleotide siRNA, the polymorphism is at, for example, 9, 10, 11, 12, 13, 14 or 16. For a 25-nucleotide siRNA, the polymorphism is at, for example, 9, 10, 11, 12, 13, 14, 15, 16 or 17. Moving the polymorphism to an off-center position may, in some instances, reduce efficiency of cleavage by the siRNA. Such compositions, i.e., less efficient compositions, may be desirable for use if off-silencing of the wild-type mRNA is detected.

4. siRNAs with single nucleotide specificity are preferably designed such that base paring at the single nucleotide in the corresponding reference (e.g., wild type) sequence is disfavored. For example, designing the siRNA such that purine:purine paring exists between the siRNA and the wild type mRNA at the single nucleotide enhances single nucleotide specificity. The purine:purine paring is selected, for example, from the group G:G, A:G, G:A and A:A pairing. Moreover, purine:pyrimidine pairing between the siRNA and the mutant mRNA at the single nucleotide enhances single nucleotide specificity. The purine:pyrimidine paring is selected, for example, from the group G:C, C:G, A:U, U:A, C:A, A:C, U:A and A:U pairing.

5. The antisense strand is designed such that perfect complementarity exists between the antisense strand of the siRNA and the target mRNA (e.g., the mutant mRNA) at the single nucleotide (e.g., the point mutation), there thus being a mismatch if the siRNA is compared (e.g., aligned) to the reference sequence (e.g., wild type allele or mRNA sequence). Preferably the siRNA is designed such that the single nucleotide (e.g., the point mutation) is at or near the intended site of cleavage. Preferably, the siRNA is designed such that single nucleotide (e.g., the point mutation) being targeted is perfectly or exactly centered in the siRNA (e.g., in the antisense strand of the siRNA). The phrase perfectly centered means that there are the same number of nucleotides flanking (i.e., 8, 9, 10, 11 or 12) the single nucleotide (e.g., the point mutation), but for any overhang, for example, a dTdT tail. For example, if a 21-nucleotide siRNA is chosen having a 2-nucleotide 3′ overhang (e.g., overhang at the 3′ end of the antisense strand), there are 9 nucleotides flanking the single nucleotide (e.g., point mutation). For a 22-nucleotide siRNA having a 2-nucleotide 3′ overhang (e.g., overhang at the 3′ end of the antisense strand) there are 9 and 10 nucleotides flanking the single nucleotide (e.g., point mutation). For a 23-nucleotide siRNA, there are 10 nucleotides flanking the single nucleotide (e.g., point mutation). For a 24-nucleotide siRNA, there are 10 and 11 nucleotides flanking the single nucleotide (e.g., point mutation). The numbers exemplified are for siRNAs having 2-nucleotide 3′ overhangs but can be readily adjusted for siRNAs having longer or shorter overhangs or no overhangs. Designing the siRNA such that the single nucleotide (e.g., point mutation is off-center with respect to the siRNA may, in some instances, reduce efficiency of cleavage by the siRNA.

siRNAs with single nucleotide specificity are preferably designed such that base paring at the single nucleotide in the corresponding reference (e.g., wild type) sequence is disfavored. For example, designing the siRNA such that purine:purine paring exists between the siRNA and the wild type mRNA at the single nucleotide enhances single nucleotide specificity. The purine:purine paring is selected, for example, from the group G:G, A:G, G:A and A:A pairing. Moreover, purine pyrimidine pairing between the siRNA and the mutant mRNA at the single nucleotide enhances single nucleotide specificity. The purine:pyrimidine paring is selected, for example, from the group G:C, C:G, A:U, U:A, C:A, A:C, U:A and A:U pairing.

6. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

7. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

8. Select one or more sequences that meet your criteria for evaluation.

Further general information about the design and use of siRNA may be found in “The siRNA User Guide,” available at The Max-Plank-Institut für Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

9. To validate the effectiveness by which siRNAs destroy mutant mRNAs (e.g., mutant huntingtin mRNA), the siRNA may be incubated with mutant cDNA (e.g., mutant huntingtin cDNA) in a Drosophila-based in vitro mRNA expression system. Radiolabeled with ³²P, newly synthesized mutant mRNAs (e.g., mutant huntingtin mRNA) are detected autoradiographically on an agarose gel. The presence of cleaved mutant mRNA indicates mRNA nuclease activity. Suitable controls include omission of siRNA and use of wild-type huntingtin cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

b) siRNA-Like Molecules

siRNA-like molecules of the invention have a sequence (i.e., have a strand having a sequence) that is “sufficiently complementary” to a target mRNA sequence to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further preferred embodiment, the “bulge” is centered at nucleotide positions 12 and 13 from the 5′end of the miRNA molecule.

c) Short Hairpin RNA (shRNA) Molecules

In certain featured embodiments, the instant invention provides shRNAs capable of mediating RNA silencing of a target sequence (e.g. target mRNA) with enhanced selectivity. In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway.

miRNAs are noncoding RNAs of approximately 22 nucleotides which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, of the invention are artificial constructs based on these naturally occurring pre-miRNAs, but which are engineered to deliver desired RNA silencing agents (e.g., siRNAs of the invention). By substituting the stem sequences of the pre-miRNA with sequence complementary to the target mRNA, a shRNA is formed. The shRNA is processed by the entire gene silencing pathway of the cell, thereby efficiently mediating RNAi.

The requisite elements of a shRNA molecule include a first portion and a second portion, having sufficient complementarity to anneal or hybridize to form a duplex or double-stranded stem portion. The two portions need not be fully or perfectly complementary. The first and second “stem” portions are connected by a portion having a sequence that, has insufficient sequence complementarity to anneal or hybridize to other portions of the shRNA. This latter portion is referred to as a “loop” portion in the shRNA molecule. The shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In shRNAs, or engineered precursor RNAs, of the instant invention, one portion of the duplex stem is a nucleic acid sequence that is complementary (or anti-sense) to the target mRNA. Preferably, one strand of the stem portion of the shRNA is sufficiently complementary (e.g., antisense) to a target RNA (e.g., mRNA) sequence to mediate degradation or cleavage of said target RNA via RNA interference (RNAi). Thus, engineered RNA precursors include a duplex stem with two portions and a loop connecting the two stem portions. The antisense portion can be on the 5′ or 3′ end of the stem. The stem portions of a shRNA are preferably about 15 to about 50 nucleotides in length. Preferably the two stem portions are about 18 or 19 to about 21, 22, 23, 24, 25, 30, 35, 37, 38, 39, or 40 or more nucleotides in length. In preferred embodiments, the length of the stem portions should be 21 nucleotides or greater. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to, and including the entire mRNA). In fact, a stem portion can include much larger sections complementary to the target mRNA (up to, and including the entire mRNA).

The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides. The overhanging nucleotides can include, for example, uracils (Us), e.g., all Us. The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop in the shRNAs or engineered RNA precursors can be 2, 3, 4, 5, 6, 7, 8, 9, or more, e.g., 15 or 20, or more nucleotides in length.

The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop portion in the shRNA can be about 2 to about 20 nucleotides in length, i.e., about 2, 3, 4, 5, 6, 7, 8, 9, or more, e.g., 15 or 20, or more nucleotides in length. A preferred loop consists of or comprises a “tetraloop” sequences. Exemplary tetraloop sequences include, but are not limited to, the sequences GNRA, where N is any nucleotide and R is a purine nucleotide, GGGG, and UUUU.

In certain embodiments, shRNAs of the invention include the sequences of a desired siRNA molecule described supra. In other embodiments, the sequence of the antisense portion of a shRNA can be designed essentially as described above or generally by selecting an 18, 19, 20, 21 nucleotide, or longer, sequence from within the target RNA (e.g., SOD1 or htt mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR, provided said portion is distant from the site of the gain-of-function mutation. This sequence can optionally follow immediately after a region of the target gene containing two adjacent AA nucleotides. The last two nucleotides of the nucleotide sequence can be selected to be UU. This 21 or so nucleotide sequence is used to create one portion of a duplex stem in the shRNA. This sequence can replace a stem portion of a wild-type pre-miRNA sequence, e.g., enzymatically, or is included in a complete sequence that is synthesized. For example, one can synthesize DNA oligonucleotides that encode the entire stem-loop engineered RNA precursor, or that encode just the portion to be inserted into the duplex stem of the precursor, and using restriction enzymes to build the engineered RNA precursor construct, e.g., from a wild-type pre-miRNA.

Engineered RNA precursors include in the duplex stem the 21-22 or so nucleotide sequences of the siRNA or siRNA-like duplex desired to be produced in vivo. Thus, the stem portion of the engineered RNA precursor includes at least 18 or 19 nucleotide pairs corresponding to the sequence of an exonic portion of the gene whose expression is to be reduced or inhibited. The two 3′ nucleotides flanking this region of the stem are chosen so as to maximize the production of the siRNA from the engineered RNA precursor and to maximize the efficacy of the resulting siRNA in targeting the corresponding mRNA for translational repression or destruction by RNAi in vivo and in vitro.

In certain embodiments, shRNAs of the invention include miRNA sequences, optionally end-modified miRNA sequences, to enhance entry into RISC. The miRNA sequence can be similar or identical to that of any naturally occurring miRNA (see e.g. The miRNA Registry; Griffiths-Jones S. Nuc. Acids Res., 2004). Over one thousand natural miRNAs have been identified to date and together they are thought to comprise ˜1% of all predicted genes in the genome. Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g. MiRScan, MiRSeeker) that predict the capability of a candidate miRNA gene to form the stem loop structure of a pri-mRNA (Grad et al., Mol. Cell., 2003; Lim et al., Genes Dev., 2003; Lim et al., Science, 2003; Lai E C et al., Genome Bio., 2003). An online registry provides a searchable database of all published miRNA sequences (The miRNA Registry at the Sanger Institute website; Griffiths-Jones S. Nuc. Acids Res., 2004). Exemplary, natural miRNAs include lin-4, let-7, miR-10, mirR-15, miR-16, miR-168, miR-175, miR-196 and their homologs, as well as other natural miRNAs from humans and certain model organisms including Drosophila melanogaster, Caenorhabditis elegans, zebrafish, Arabidopsis thalania, mouse, and rat as described in International PCT Publication No. WO 03/029459.

Naturally-occurring miRNAs are expressed by endogenous genes in vivo and are processed from a hairpin or stem-loop precursor (pre-miRNA or pri-miRNAs) by Dicer or other RNAses (Lagos-Quintana et al., Science, 2001; Lau et al., Science, 2001; Lee and Ambros, Science, 2001; Lagos-Quintana et al., Curr. Biol., 2002; Mourelatos et al., Genes Dev., 2002; Reinhart et al., Science, 2002; Ambros et al., Curr. Biol., 2003; Brennecke et al., 2003; Lagos-Quintana et al., RNA, 2003; Lim et al., Genes Dev., 2003; Lim et al., Science, 2003). miRNAs can exist transiently in vivo as a double-stranded duplex but only one strand is taken up by the RISC complex to direct gene silencing. Certain miRNAs, e.g. plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between an miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism. In particular embodiments, the miRNA sequence is that of a naturally-occurring miRNA sequence, the aberrant expression or activity of which is correlated with a miRNA disorder.

d) Dual Functional Oligonucleotide Tethers

In other embodiments, the RNA silencing agents of the present invention include dual functional oligonucleotide tethers useful for the intercellular recruitment of a miRNA. Animal cells express a range of miRNAs, noncoding RNAs of approximately 22 nucleotides which can regulate gene expression at the post transcriptional or translational level. By binding a miRNA bound to RISC and recruiting it to a target mRNA, a dual functional oligonucleotide tether can repress the expression of genes involved e.g., in the arteriosclerotic process. The use of oligonucleotide tethers offer several advantages over existing techniques to repress the expression of a particular gene. First, the methods described herein allow an endogenous molecule (often present in abundance), an miRNA, to mediate RNA silencing; accordingly the methods described herein obviate the need to introduce foreign molecules (e.g., siRNAs) to mediate RNA silencing. Second, the RNA-silencing agents and, in particular, the linking moiety (e.g., oligonucleotides such as the 2′-O-methyl oligonucleotide), can be made stable and resistant to nuclease activity. As a result, the tethers of the present invention can be designed for direct delivery, obviating the need for indirect delivery (e.g. viral) of a precursor molecule or plasmid designed to make the desired agent within the cell. Third, tethers and their respective moieties, can be designed to conform to specific mRNA sites and specific miRNAs. The designs can be cell and gene product specific. Fourth, the methods disclosed herein leave the mRNA intact, allowing one skilled in the art to block protein synthesis in short pulses using the cell's own machinery. As a result, these methods of RNA silencing are highly regulatable.

The dual functional oligonucleotide tethers (“tethers”) of the invention are designed such that they recruit miRNAs (e.g., endogenous cellular miRNAs) to a target mRNA so as to induce the modulation of a gene of interest. In preferred embodiments, the tethers have the formula T-L-μ, wherein T is an mRNA targeting moiety, L is a linking moiety, and μ is an miRNA recruiting moiety. Any one or more moiety may be double stranded. Preferably, however, each moiety is single stranded.

Moieties within the tethers can be arranged or linked (in the 5′ to 3′ direction) as depicted in the formula T-L-μ (i.e., the 3′ end of the targeting moiety linked to the 5′ end of the linking moiety and the 3′ end of the linking moiety linked to the 5′ end of the miRNA recruiting moiety). Alternatively, the moieties can be arranged or linked in the tether as follows: μ-T-L (i.e., the 3′ end of the miRNA recruiting moiety linked to the 5′ end of the linking moiety and the 3′ end of the linking moiety linked to the 5′ end of the targeting moiety).

The mRNA targeting moiety, as described above, is capable of capturing a specific target mRNA. According to the invention, expression of the target mRNA is undesirable, and, thus, translational repression of the mRNA is desired. The mRNA targeting moiety should be of sufficient size to effectively bind the target mRNA. The length of the targeting moiety will vary greatly depending, in part, on the length of the target mRNA and the degree of complementarity between the target mRNA and the targeting moiety. In various embodiments, the targeting moiety is less than about 200, 100, 50, 30, 25, 20, 19, 18, 17, 16, 15, 14, 13, 12, 11, 10, 9, 8, 7, 6, or 5 nucleotides in length. In a particular embodiment, the targeting moiety is about 15 to about 25 nucleotides in length.

The miRNA recruiting moiety, as described above, is capable of associating with an miRNA. According to the invention, the miRNA may be any miRNA capable of repressing the target mRNA. Mammals are reported to have over 250 endogenous miRNAs (Lagos-Quintana et al. (2002) Current Biol. 12:735-739; Lagos-Quintana et al. (2001) Science 294:858-862; and Lim et al. (2003) Science 299:1540). In various embodiments, the miRNA may be any art-recognized miRNA.

The linking moiety is any agent capable of linking the targeting moieties such that the activity of the targeting moieties is maintained. Linking moieties are preferably oligonucleotide moieties comprising a sufficient number of nucleotides such that the targeting agents can sufficiently interact with their respective targets. Linking moieties have little or no sequence homology with cellular mRNA or miRNA sequences. Exemplary linking moieties include one or more 2′-O-methylnucleotides, e.g., 2′-O-methyladenosine, 2′-O-methylthymidine, 2′-O-methylguanosine or 2′-O-methyluridine.

II. Methods for Synthesizing RNA Silencing Agents with Enhanced Target Discrimination

A. Designing RNA Silencing Agents with Optimally Positioned Specificity-Determining Residues

In certain aspects, the invention provides methods for synthesizing optimized RNA silencing agents capable of enhanced discriminatory RNA silencing wherein the specificity-determining residue(s) of the RNA silencing agent (e.g. an siRNA) is positioned distal to the “seed region” (also known as the “seed sequence”) of RNA silencing agent. As is well-known in the art, the “seed region” of an RNA silencing agent consists of the sequence formed by nucleotide positions 2-7 or 2-8 from the 5′ terminal nucleotide of the antisense strand of the RNA silencing agent (e.g., the guide strand of an siRNA) (see, e.g., Lewis B P et al., Cell, (2005), 120: 15; Brennecke et al., PloS Biology, (2005), 3: e85). The seed sequence is generally considered to be most critical for target selectivity. Surprisingly, however, the invention demonstrates that positioning the specificity-determining nucleotide in the 3′ end or central region (preferably a position in the 3′ side of the seed sequence) of the antisense strand of the RNA silencing agent imparts improved discriminatory RNA silencing. The specificity determining residue of the antisense strand is preferably positioned such that it forms a complementary base pair (e.g., a Watson-Crick base pair) with the target mRNA (e.g., a disease-associated allelic polymorphism), while forming a nucleotide mismatch or wobble base pair with the reference, non-target RNA (e.g., a wild-type allelic sequence).

In other aspects, the invention is directed to methods of enhancing discriminatory RNA silencing of an RNA silencing agent comprising positioning the specificity-determining residue at a position which is 3′ of the seed sequence. The specificity-determining residue is preferably positioned by introducing a nucleotide mismatch or wobble base pair within the central region or 3′ end of the antisense strand of said agent, wherein the mismatch or wobble is between the antisense strand of the RNA silencing agent and non-target RNA (e.g. RNA corresponding to a wild-type allele of a gain-of-function protein or a non-target SNP allele).

In one aspect, the invention provides an RNA silencing agent capable of enhanced discriminatory RNA silencing wherein the RNA silencing agent comprises a specificity determining-nucleotide at a position which is 3′ of the seed sequence of the agent (e.g., in the central or 3′end of the antisense strand of said agent). Preferably, the specificity-determining nucleotide forms a complementary base pair (i.e., Watson-Crick base pair) with the target site of a target mRNA (e.g., an allelic polymorphism, e.g., a disease-associated SNP), and a mismatched or wobble base pair with a reference, non-target RNA (e.g., an mRNA corresponding to the wild-type allele of the allelic polymorphism). In certain embodiments, the RNA silencing agent is a siRNA. In other embodiments, the RNA silencing agent is a miRNA, shRNA, or a dual-function oligonucleotide.

In preferred embodiments, the RNA silencing agent comprises a specificity-determining nucleotide located at a nucleotide position selected from the group consisting of P8, P9, P10, P12, P13, P14, P15, P16 and P19, wherein the nucleotide position is relative to the 5′ end (ie. 5′ terminus) of the antisense strand. For example, nucleotide position P8 corresponds to the position of the nucleotide located eight nucleotides from the 5′ terminus of the antisense strand. In the case of a shRNA, the nucleotide position P8 of the shRNA antisense strand (ie. antisense stem portion) corresponds to the P8 position of the antisense strand of the siRNA that is generated by cleavage of said shRNA. In the case of a dual-function oligonucleotide, nucleotide position P8 of the antisense strand corresponds to nucleotide position P8 from the 5′ terminal nucleotide of the mRNA targeting moiety (T).

In other preferred embodiments, the RNA silencing agent comprises a specificity-determining nucleotide located at a nucleotide position selected from the group consisting of P9, P10, P12, P13, P14, and P16, wherein the nucleotide position is relative to the 5′end of the antisense strand.

In particularly preferred embodiments, the specificity-determining nucleotide is located at nucleotide position 10 (P10) relative to the 5′ terminus (or 5′ end) of the antisense strand. In another particularly preferred embodiment, the specificity-determining nucleotide is located at nucleotide position 16 (P16) relative to the 5′ terminus (or 5′end) of the antisense strand.

In another embodiment, an RNA silencing agent of the invention discriminates against a non-target RNA that is encoded by a wild-type allele corresponding to the mutant allele of a gene encoding a mutant gain-of-function protein which is, in turn, targeted by the RNA silencing agent of the invention. In one embodiment, said mutant gain-of-function protein is a mutant SOD1 protein. In another embodiment, said mutant gain-of-function protein is a mutant Huntingtin protein.

In other embodiments, the specificity-determining nucleotide is complementary to the to the target region of target mRNA (e.g., an allelic polymorphism), while forming a wobble base pair (e.g., a G:U or U:G base pair) with the non-target, reference mRNA (e.g., a wild-type allele corresponding to the allelic polymorphism). In more preferred embodiments, the specificity-determining nucleotide forms Watson-Crick base base pair with the target region of the target mRNA, but forms a mismatched base pair with the non-target mRNA. In one embodiment, the mismatch is a purine:pyrimidine mismatch (e.g., A:C or C:A) between the specificity-determining nucleotide and a nucleotide of a non-target mRNA (ie. the nucleotide corresponding to a polymorphic nucleotide of the target mRNA, e.g, a single nucleotide polymorphism). In another embodiment, the mismatch is a pyrimidine:pyrimidine mismatch (e.g., C:C, C:U, U:U or U:C). More preferably, the mismatch is a purine:purine mismatch with the non-target mRNA (e.g., G:G, A:G, A:A, or G:A). In one preferred embodiment, the mismatch is a G:G mismatch. In another preferred embodiment, the mismatch is a G:A mismatch.

In other embodiments, the RNA silencing agent provides more than 4-fold discrimination in RNA silencing activity between two alleles (e.g., a wild-type and polymorphic allele) which differ by at least one nucleotide (e.g. 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, or 200-fold). In yet other embodiments, the RNA silencing agent provides at least 20-fold discrimination between two alleles which differ by only a few nucleotides (e.g., a single nucleotide).

In other embodiments of the invention, an RNA silencing agent (or any portion thereof) of the invention as described supra may be modified such that the activity of the agent is further improved. For example, the RNA silencing agents described in Section II A supra may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

In certain embodiments, the optimized RNA silencing agents of the invention may be substituted with a destabilizing nucleotide to further enhance single nucleotide target discrimination (see Section II-B infra). Such a modification may be sufficient to abolish the specificity of the RNA silencing agent for a non-target mRNA (e.g. wild-type mRNA), without appreciably affecting the specificity of the RNA silencing agent for a target mRNA (e.g. gain-of-function mutant mRNA). In one exemplary embodiment, a RNA silencing agent is optimized both by (i) positioning the specificity determining nucleotide at a position distal to the seed sequence (e.g., P10 or P16); and (ii) substituting at least one nucleotide adjacent to the specificity-determining nucleotide (e.g., within 1, 2, 3, 4, or 5 nucleotides) with a destabilizing nucleotide (e.g., a universal nucleotide, e.g. an inosine or analog thereof).

B. Modified RNA Silencing Agents with Destabilizing Nucleotides

In certain embodiments, the invention provides RNA silencing agents which are modified by substituting at least one nucleotide in an antisense strand of the RNA silencing agent with a destabilizing nucleotide in order to enhance the ability of the RNA silencing agent to discriminate among a target mRNA and a non-target mRNA that differ in sequence by at least one nucleotide. In certain embodiments, the introduction of a destabilizing nucleotide in the antisense strand (or guide strand) of the RNA silencing agent serves to lower the melting temperature of duplex formed by the antisense strand of the RNA silencing agent and its target. In preferred embodiments, the decrease in melting temperature is sufficient to abolish or diminish the specificity of the RNA silencing agent for a non-target mRNA, without appreciably affecting the specificity of the RNA silencing agent for a target mRNA. The modified RNA silencing agents of the invention are therefore capable of silencing expression of target mRNA without sufficiently silencing the expression of a non-target mRNA (e.g. wild-type mRNA).

In certain embodiments, the RNA silencing agents of the invention are modified by the introduction of a destabilizing nucleotide in the antisense strand of said RNA silencing agent wherein the destabilizing nucleotide forms a mismatched base pair with the corresponding nucleotide in the sense strand of the RNA silencing agent. Preferably, the mismatched base pair is selected from the group consisting of G:A, C:A, C:U, G:G, A:A, C:C and U:U. More preferably, the mismatched base pair is selected from the group consisting of G:A, G;G, A:A, A:G. In a related embodiment, an RNA silencing agent of the invention is modified by the introduction of at least one destabilizing nucleotide in the antisense strand of said RNA silencing agent such that a wobble base (e.g., G:U) is formed with the corresponding sense strand. It is anticipated that the destabilizing nucleotide in the antisense strand (or guide strand) of the RNA silencing agent will form a mismatch or wobble base pair with the opposing nucleotide in the target or non-target mRNA, thereby decreasing the melting temperature of duplex such that silencing of the non-target mRNA is abolished or diminished, while silencing of the target mRNA is maintained.

In preferred embodiments the RNA silencing agents of the invention are modified by the introduction of at least one universal nucleotide in the antisense strand thereof. Universal nucleotides comprise base portions that are capable of base pairing indiscriminately with any of the four conventional nucleotide bases (e.g. A,G,C,U). A universal nucleotide is preferred because it has relatively minor effect on the stability of the RNA duplex or the duplex formed by the guide strand of the RNA silencing agent and the target mRNA. In one embodiment, the universal nucleotide does not base pair to a significant degree with any base on the complementary strand of the RNA silencing agent. In certain preferred embodiments, the universal base may base pair with a nucleotide base on a complementary polynucleotide, but does not base pair in a significantly different way with different bases placed in the same position. More preferably, the universal nucleotide can base pair equally well with each of the natural bases of RNA (ie. A, G, C, U) when placed opposite them in a RNA silencing agent.

In some embodiments, the RNA silencing agents of the invention comprise a universal nucleotide having a base portion selected from the group consisting of 5-nitroindole (also known as 5-nitro, 1-(β-D-2-deoxyribofuranosyl; see e.g. Loakes and Brown, Nucleic Acids Res., 22: 4039-43, (1994)), 4′-nitroindole, 6′-nitroindole, 7-azaindole, 6-methyl-7-azylindole, proynyl-7-azaindole, 3-nitropyrrole (also known as 1-(2′-deoxy-β-D-2-ribofuranosyl)-3-nitropyrrole; see Nichols et al., Nature, 396:492-3 (1994)), pyrrollpyrizine, imidizopyridine, isocarbostyril, 3-methyl isocarbostyril, 5-methyl isocarbostyril, propynylisocarbostyril, 3-methyl-7-probynyl isocarbostyril, 5-propynyl uracil, 2-thio-5-propynyl uracil, 2-thio-thymine, 2-thio-uracil, 7-deaza-guanine, 7-deaza-8aza-guanine, 2,6-diaminopurine, allenyl-7-azaindole, 3′-nitroazole, 4′-nitrobenzimidazole, nitroindazole (e.g., 5′nitroindazole), 4-aminobenzimidazole, imidazo-4,5-dicarboxamide, 3′-nitroimidazole, imidazole-4′carboxamide, 3-(4-nitroazol-1-yl)-1,2-propanediol, 8-aza-7-deazaadenine, (pyrazolo[3,4-d]pyrimidin-4-amine), and nebularine. In other embodiments, the RNA silencing agents of the invention may comprise a universal nucleotide comprising a nucleoside portion selected from the group of propynyl derivatives consisting of 8-aza-7-deaza-guanosine, 8-aza-7-deaza-2′-deoxyadenosine, 2′-deoxycytidine, 2′-deoxyuridine, 2′-deoxyadenosine, 2′-deoxyguanosine, and pyrrolo[2,3-d]pyrmidine nucleosides.

In certain preferred embodiments, the RNA silencing agents of the invention comprise a nucleotide having an inosine base portion or an inosine analog selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine.

In particularly preferred embodiments, the RNA silencing agent of the invention is substituted with an inosine residue or a naturally occurring analog thereof. Inosines form stable base pairs with all four conventional ribonucleotides and the strength of the base pairing is approximately equal in each case (Ohtsuka, J. Biol. Chem., 260(5): 2605-8 (1985)).

The modified RNA silencing agents of the invention are particularly advantageous where the unmodified RNA silencing agent (ie. the antisense strand of the RNA silencing agent) or its target has a G/C content of at least 35% or greater. Unmodified RNA silencing agents with such a high G/C content may form more stable, sequence-mediated, interactions with a non-target mRNA and thereby causing undesirable silencing of the non-target mRNA. The modified RNA silencing agents of the invention comprise destabilizing nucleotides which counteract undesirable interactions with non-target mRNA. Accordingly, in exemplary embodiments, a modified RNA silencing agent of the invention (or its target mRNA) has a G/C content of greater than 35%. Greater than 40% identity, e.g., 40%, 41%, 42%, 43%, 44%, 45%, 46%, 47%, 48%, 49%, 50%, 51%, 52%, 53%, 54%, 55%, 56%, 57%, 58%, 59%, or even 60% G/C content, is contemplated.

In certain embodiments, an RNA silencing agent of the invention comprises about 1 to about 10 destabilizing nucleotides in the antisense strand. In more preferred embodiments, an RNA silencing agent of the invention comprises about 1 to about 8 destabilizing nucleotides in the antisense strand. In yet more preferred embodiments, an RNA silencing agent of the invention comprises about 1 to about 5 destabilizing nucleotides in the antisense strand. In still more preferred embodiments, an RNA silencing agent of the invention comprises about 1 to about 3 destabilizing nucleotides (e.g. 2 destabilizing nucleotides) in the antisense strand.

The RNA silencing agents of the invention are preferably modified by the introduction of at least one destabilizing nucleotide within 5 nucleotides from the specificity-determining nucleotide of the RNA silencing agent. For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (ie. such that there are 2 stabilizing nucleotides between the destabilizing nucleotide and the specificity-determining nucleotide). In RNA silencing agents having two strands or strand portions (e.g. siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

In other exemplary embodiments, the modified RNA silencing agents of the invention comprise two or more destabilizing nucleotides in the antisense strand thereof, wherein at least one destabilizing nucleotide is on the 3′ side of the specificity-determining nucleotide and at least one destabilizing nucleotide on the 5′side of the specificity-determining nucleotide. In certain embodiments, at least one specificity-determining nucleotide is perfectly centered between two destabilizing nucleotides. In other embodiments, the specificity-determining nucleotide is at a position which is off-center between two or more destabilizing nucleotides. In an exemplary embodiment, a modified RNA silencing agent of the invention comprises a specificity-determining nucleotide which is perfectly centered between two destabilizing nucleotides (e.g. two universal nucleotides) such that each destabilizing nucleotide is at a nucleotide position which is three nucleotides from the specificity-determining nucleotide.

In other embodiments, the modified RNA silencing agents of the invention comprise two or more destabilizing nucleotides which are separated from each other by less than 10 stabilizing nucleotides. For example, an RNA silencing agent of the invention may comprise two destabilizing nucleotides which are separated by 9, 8, 7, 6, 5, 4, 3, 2, or 1 nucleotide(s). In a preferred embodiment, the two destabilizing nucleotides are separated from each other by at least one stabilizing nucleotide (e.g. the specificity-determining nucleotide). More preferably, the destabilizing nucleotides are separated by at least two nucleotides. In a particularly preferred embodiment, the destabilizing nucleotides are separated by five stabilizing nucleotides.

Introduction of a destabilizing nucleotide preferably has minor effects on the stability or melting temperature of the duplex formed by the guide strand of the RNA silencing agent and its target mRNA. Minor effects are preferred in order to retain the ability of the guide strand to form a stable binding interaction with the target mRNA, while largely abolishing the ability of the guide strand to form a stable binding interaction with the non-target mRNA. The effects of the destabilizing nucleotide on the stability the of the duplex formed by the guide strand of the RNA silencing agent may inferred by the effects of the destabilizing nucleotide on the stability of the duplex formed by the sense and antisense strands of the RNA silencing agent. In certain embodiments, the destabilizing nucleotide may be effective to reduce the Tm of either duplex by less than 10° C. In other embodiments, the destabilizing nucleotide may be effective to reduce the Tm of the duplex by less than 8° C. In other embodiments, the destabilizing nucleotide may be effective to reduce the Tm of the duplex by less than 6° C. In other embodiments, the destabilizing nucleotide may be effective to reduce the Tm by less than 4° C. In other embodiments, the destabilizing nucleotide may be effective to reduce the Tm by less than 2° C. In other embodiments, the destabilizing nucleotide may be effective to reduce the Tm by less than 1° C. Groups or combinations of destabilizing nucleotides may be similarly effective. Such reduction in Tm may be the result of reduced amounts of destabilization of hybridization of less than 50 kcal/mol, less than about 25 kcal/mol, less than about 15 kcal/mol, less than about 5 kcal/mol, or less than about 2 kcal/mol.

In preferred embodiments, the stability difference (ie. the difference in melting temperature (ΔTm)) between the melting temperature of a duplex of a modified RNA silencing agent or that formed by the guide strand thereof and its target (Tm1) and the melting temperature of a duplex of the reference RNA silencing agent or that formed by the guide strand thereof and its target (Tm2) is small. In one embodiment, the stability difference is less than modified RNA silencing agent and the reference RNA silencing agent is small. In one embodiment, the stability difference is less than 10° C. In other embodiments, the stability difference is less than 8° C. In more preferred embodiments, the stability difference is less than 6° C. In still more preferred embodiments, the stability difference less than 4° C. In yet more preferred embodiment, stability difference is less than 2° C. In still more preferred embodiments, the stability difference is less than 1° C. The stability difference may be expressed as a free energy difference (ΔG). In preferred embodiments, the free energy difference may be less than 50 kcal/mol, less than about 25 kcal/mol, less than about 15 kcal/mol, less than about 5 kcal/mol, or less than about 2 kcal/mol.

The melting temperature of a duplex can be calculated (e.g. manually or in silico) using any of the formulas described supra. However, when calculating the Tm of the modified RNA silencing agents of the invention, the number of destabilizing nucleotides in the duplex of RNA silencing agent are subtracted from the total number of nucleotides in the duplex; and the Tm of a duplex containing the remaining number of nucleotides is then calculated.

The melting temperature of a duplex can also be determined experimentally by hybridization assay which comprise covalently linking a target sequence to a substrate (e.g. a nitrocellulose filter or membrane), subjecting the target sequence to hybridization with a complementary probe strand that is detectably labeled (e.g. with a radioactive isotope, a fluorescent dye, or a reactive compound) such that a duplex is formed, washing the substrate at an increased temperature or reduced salt concentrations to remove unhybridized probe strand, and quantifying the remaining amount of labeled probe strand. The melting temperature may be determined to be the washing temperature at which approximately 50% of the probe strand remains associated with the target sequence. In order to determine the melting temperature of duplex portion of an RNA silencing agent, either sense or antisense strand is labeled and the corresponding antisense or sense strand is linked to the substrate. Alternatively, to determine the melting temperature of the duplex formed by the guide strand of the siRNA and the target mRNA, either the guide strand or target mRNA is labeled and the respective target mRNA or guide strand is linked to the substrate. Advantageously the hybridization is effected in aqueous solution at an elevated temperature. Alternatively hybridization may be effected at a lower temperature in the presence of an organic solvent which is effective to destabilize the hybrid, such as a solvent containing formamide. Other hybridization conditions may be altered such a pH and ionic strength may be varied. The detailed conditions for hybridization can be found in Sambrook et al., Molecular Cloning: A Laboratory Manual, Cold Spring Harbor Laboratory Press, Cold Spring Harbor, N.Y. (2001), and Anderson, Nucleic Acid Hybridization, Springer-Verlag New York Inc., N.Y. (1999).

An additional method for determining melting temperature involves the use of UV spectrophotometry whereby the concentration of single and double-stranded RNA molecules is determined from high-temperature absorbance at 280 nm (see, e.g. Serra et al., Nuc. Acids Res., 32(5): 1824-8, (2004)). Other methods for calculating or experimentally determining Tm are known in the art (see, e.g., Breslauer et al., Proc. Natl. Acad. Sci. USA 83: 3746-50 (1986); Baldino et al., Methods in Enzymol., 168: 761-77, (1989); and Breslauer, Methods in Enzymol. 259: 221-242 (1995)).

As an alternative to melting temperature, the stability of the duplex portion of an RNA silencing agent or the duplex formed by the guide strand of the siRNA and the target mRNA may be determined by measuring or calculating the average free energy of the duplex. Methods for determining free energy include the “nearest neighbor” method described by Freier et al., Proc. Natl. Acad. Sci. USA, 83(24): 9373-7).

In other embodiments, the discriminatory RNA silencing activity of a modified RNA silencing agent can be directly determined using a biochemical or in vitro based assay which recapitulates the RNA silencing pathways of interest (e.g. an assay comprising biochemical components necessary for RNAi). For example, an RNA silencing agent of the invention can be introduced into a Drosophila embryo lysate or transfected into a whole cell (e.g. human cell) wherein the lysate or cell further comprises a target mRNA and non-target mRNA. The ability of the RNA silencing agent is capable of preferentially silencing the target mRNA over the non-target mRNA can be determined by labeling the respective target and non-target mRNA with different detectable labels (e.g. different fluorophores emitting light at different visible wavelength) such that the silencing of each mRNA can be distinguished quantitatively. Alternatively, the discriminatory RNA silencing activity can be determined by measuring the ability of the RNA silencing agent to silence target and non-target mRNAs in separate experiments. In one exemplary embodiment, the relevant portion of each target mRNA or non-target mRNA is operably linked to a sequence coding for a reporter protein (a luciferase). These hybrid mRNAs are therefore capable of coding for a fusion protein having an activity that may be readily quantified. In another exemplary embodiment, each target mRNA or non-target mRNA can be 5′ radiolabeled (e.g using guanylyl transferase as described in Tuschl et al., 1999, supra and references therein) and introduced to the assay system (e.g. an RNAi-competent cell or lysate) in the presence of the RNA silencing agent. The products of the in vitro reaction are then isolated and analyzed on a denaturing acrylamide or agarose gel to determine if the target or non-target mRNA has been cleaved in response to the presence of the engineered RNA precursor in the reaction.

The discriminatory RNA silencing activity of an RNA silencing agent of the invention can be compared with that of an unmodified RNA silencing agent to determine the degree of enhancement. In preferred embodiments the modified RNA silencing agent is capable of (i) substantially silencing the target sequence, and (ii) not substantially silencing non-target sequence at a concentration greater than 0.05 nM. In a more preferred embodiment the modified RNA silencing agent is capable of (i) substantially silencing the target sequence, and (ii) not substantially silencing non-target sequence at a concentration greater than 0.5 nM. In a still more preferred embodiment, the modified RNA silencing agent is capable of (i) substantially silencing the target sequence, and (ii) not substantially silencing non-target sequence at a concentration greater than 5 nM. In yet a more preferred embodiment, the RNA silencing agent is capable of (i) substantially silencing the target sequence, and (ii) not substantially silencing non-target sequence at a concentration greater than 50 nM.

IV. Other Modifications for RNA Silencing Agents

In certain embodiments, the modifications described supra may be introduced in an RNA silencing agent in combination with one or more of the following modifications. The modifications described infra serve to further enhance the activity of the RNA silencing agents of the invention. In certain aspects of the invention, an RNA silencing agent (or any portion thereof) of the invention as described supra may be modified such that the in vivo activity of the agent is improved without compromising the agent's RNA silencing activity. The modifications can, in part, serve to enhance the efficacy, to improve stability of the agent (e.g., to prevent degradation), to promote cellular uptake, to enhance the target efficiency, to improve patient tolerance to the agent, and/or to reduce toxicity.

1) RNA Silencing Agents with Enhanced Efficacy and Specificity

In certain embodiments, the RNA silencing agents of the invention have been altered to facilitate enhanced efficacy and specificity in mediating RNAi according to asymmetry design rules (see International Publication No. WO 2005/001045, US Publication No. 2005-0181382 A1). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the invention may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an RNA silencing agent of the invention may be enhanced such that there is at least one base pair comprising a modified nucleotide. In preferred embodiments, the modified nucleotide is selected from the group consisting of 2-amino-G, 2-amino-A, 2,6-diamino-G, and 2,6-diamino-A.

2) RNA Silencing Agents with Enhanced Stability

The RNA silencing agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention the RNA silencing agents may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphorthioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents of the instant invention. Additional modified residues include, deoxy-abasic, inosine, N3-methyl-uridine, N6, N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby preorganizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the invention includes RNA silencing agents having two complementary strands of nucleic acid, wherein the two strands are crosslinked. The invention also includes RNA silencing agents which are conjugated or unconjugated (e.g., at its 3′ terminus) to another moiety (e.g. a non-nucleic acid moiety such as a peptide), an organic compound (e.g., a dye, cholesterol), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention include both unmodified siRNAs and modified siRNAs as known in the art, such as crosslinked siRNA derivatives or derivatives having non nucleotide moieties linked, for example to their 3′ or 5′ ends. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The RNA silencing agents of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The RNA silencing agents of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the agent can be radiolabeled, e.g., using ³H, ³²P, or other appropriate isotope.

V. Target mRNAs of RNA Silencing Agents

In one embodiment, the target mRNA of the invention specifies the amino acid sequence of a cellular protein (e.g., a nuclear, cytoplasmic, transmembrane, or membrane-associated protein). In another embodiment, the target mRNA of the invention specifies the amino acid sequence of an extracellular protein (e.g., an extracellular matrix protein or secreted protein). As used herein, the phrase “specifies the amino acid sequence” of a protein means that the mRNA sequence is translated into the amino acid sequence according to the rules of the genetic code. The following classes of proteins are listed for illustrative purposes: developmental proteins (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogene-encoded proteins (e.g., ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES); tumor suppressor proteins (e.g., APC, BRCA1, BRCA2, MADH4, MCC, NF I, NF2, RB I, TP53, and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextriinases, DNA and RNA polymerases, galactosidases, glucanases, glucose oxidases, granule-bound starch synthases, GTPases, helicases, hernicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

In certain aspects of the invention, the target mRNA molecule of the invention specifies the amino acid sequence of a protein associated with a pathological condition. For example, the protein may be a pathogen-associated protein (e.g., a viral protein involved in immunosuppression of the host, replication of the pathogen, transmission of the pathogen, or maintenance of the infection), or a host protein which facilitates entry of the pathogen into the host, drug metabolism by the pathogen or host, replication or integration of the pathogen's genome, establishment or spread of infection in the host, or assembly of the next generation of pathogen. Alternatively, the protein may be a tumor-associated protein or an autoimmune disease-associated protein.

In one embodiment, the target mRNA molecule of the invention specifies the amino acid sequence of an endogenous protein (i.e., a protein present in the genome of a cell or organism, e.g., a mammalian cell or organism). In another embodiment, the target mRNA molecule of the invention specified the amino acid sequence of a heterologous protein expressed in a recombinant cell or a genetically altered organism e.g., a recombinant mammalian cell or organism. In another embodiment, the target mRNA molecule of the invention specified the amino acid sequence of a protein encoded by a transgene (i.e., a gene construct inserted at an ectopic site in the genome of the cell, e.g. a mammalian cell). In yet another embodiment, the target mRNA molecule of the invention specifies the amino acid sequence of a protein encoded by a pathogen genome which is capable of infecting a cell or an organism (e.g., a mammalian cell or organism) from which the cell is derived.

In certain exemplary aspects, the target mRNA molecule of the invention comprises a polymorphism or mutation but a sequence with a high degree of overall sequence identity (e.g. 80%, 85%, 90%, 95%, 98%, or greater,) with a second, non-target, mRNA that lacks the polymorphism or mutation. In certain embodiments, the target mRNA is encoded by the same gene that encodes the non-target mRNA. In other embodiments, the target mRNA is encoded by a different gene than that which encodes the non-target mRNA. In certain embodiments, the target mRNA has a high degree of sequence identity with a non-target mRNA that encodes a protein having a different function that the protein encoded by the target mRNA. In other embodiments, the target mRNA encodes a protein which performs the same biochemical function as the protein encoded by the non-target mRNA. In exemplary embodiments, the target mRNA comprises an allelic polymorphism or mutation (e.g., a single nucleotide polymorphism) that is specific to a particular allele of a gene (e.g., a disease-associated allele) and the non-target mRNA is encoded by a second allele (e.g. the wild-type allele) of the same gene. Accordingly, an object of the invention is to silence the expression of target mRNA which are associated with diseases or disorders (e.g. gain-of-function disorders), without substantially silencing the expression of a non-target mRNA (e.g., the corresponding wild-type mRNA).

I. Target mRNAs Associated with Gain-of-Function Disorders

The term “gain-of-function mutation” as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene which gives rise to the change in the function of the encoded protein. In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein. Gain-of-function mutations may give rise to gain-of-function diseases or disorders, including neurodegenerative disease.

As used herein, the term “gain-of-function disorder”, refers to a disorder characterized by a gain-of-function mutation. In one embodiment, the gain-of-function disorder is a neurodegenerative disease caused by a gain-of-function mutation, For example, Amyotrophic Lateral Sclerosis, Alzheimer's disease, Huntington's disease, and Parkinson's disease are associated with gain-of-function mutations in the genes encoding SOD1 (see Rosen et al., Nature, 362, 59-62, 1993; Rowland, Proc. Natl. Acad. Sci. USA, 92, 1251-1253, 1995), Amyloid Precursor Protein or APP (see Ikezu et al, EMBO J., (1996), 15(10):2468-75), Huntingtin or htt (see Rubinsztein, Trends Genet., (2002), 18(4):202-9), and alpha-synuclein (see, for example, Cuervo et al., Science, (2004), 305(5688): 1292-5), respectively. In another embodiment, the gain-of-function disorder is caused by a gain-of-function mutation in an oncogene, e.g., cancers caused by a mutation in the ret oncogene (e.g., ret-1), for example, gastrointestinal cancers, endocrine tumors, medullary thyroid tumors, parathyroid hormone tumors, multiple endocrine neoplasia type 2, and the like. Additional exemplary gain-of-function disorders include Alzheimer's, human immunodeficiency disorder (HIV), and slow channel congenital myasthenic syndrome (SCCMS), spinocerebellar ataxia type 3, and sickle cell anemia.

The compositions of the invention are particularly well-suited for silencing the expression of gain-of-function disorders characterized by polymorphic regions (i.e., regions containing allele-specific or allelic polymorphisms, e.g. single-nucleotide polymorphisms (SNPs)) or point mutations (e.g. a point mutation occurring in a single allele in the mutant gene) where silencing the expression of the mutant allele, but not the wild type allele, is required. In a particularly preferred embodiment, the RNA silencing agents of the invention are capable of allelic discrimination with single nucleotide specificity.

In one exemplary embodiment, the RNA silencing agents of the invention target ALS-associated SOD1 single nucleotide point mutations which result in single amino acid changes in SOD1 protein (ALS online database for ALS genetic (SOD1, ALS and other) mutations. In one embodiment, the RNA silencing agent is designed to target the Arg4Val mutation, which is the most common substitution in SOD1 and occurs in 50 percent of American patients with type 1 ALS. In another embodiment, an RNA silencing agent of the invention is designed to target the Gly37Arg mutation, which is associated with early onset of the disease but a longer survival time. In another embodiment, an RNA silencing agent of the invention is designed to target the CNTF gene, which appears to accelerate the onset of the disease. The CNTF mutation alone has no ill effects, but in combination with the SOD1 mutation, disease symptoms appear decades earlier compared to other affected family members. Other point mutations which may be targeted by the RNA silencing agents of the invention include the point mutations listed in Table 1 of International Publication No. WO 2004/042027 which is incorporated by reference herein.

In other embodiments, the RNA silencing agents of the invention target polymorphisms (e.g. single nucleotide polymorphisms) in the gain-of-function gene which is associated with a trinucleotide repeat disease such polyglutamine repeat diseases. Polyglutamine diseases have an expanded CAG repeat region in one allele as the genetic change. Since over 80 normal genes with CAG repeat regions are known to exist in cells, siRNAs targeting these CAG repeats cannot be used without risking widespread destruction of normal CAG repeat-containing mRNAs. Accordingly, said RNA silencing agents preferably target selected polymorphic regions (i.e., regions containing allele-specific or allelic polymorphisms) which are distinct from the site of mutation in the genes encoding mutant proteins. In particular, an RNA silencing agent of the invention may be designed to target a polymorphism in a target mRNA that encodes a dominant, gain-of-function mutant protein associated with a trinucleotide repeat disorder, including without limitation Huntington's disease (Huntingtin protein), spino-cerebellar ataxia type 1 (Ataxin 1), spino-cerebellar ataxia type 2 (Ataxin-2), spino-cerebellar ataxia type 3 (Ataxin-3), spino-cerebellar ataxia type 6 (α_{1A}-voltage-dependent calcium channel subunit), spino-cerebellar ataxia type 7 (Ataxin-7), spinal bulbar muscular atrophy (Androgen receptor (AR)), dentatoiubral-pallidoluysian atrophy (Atrophin-1), or other diseases characterized by the presence of trinucleotide repeats.

In an exemplary embodiment, the RNA silencing agents of the invention may be designed to target an allelic polymorphism (P) within the gene encoding, for example, a mutant human huntingtin protein (htt) for the treatment of Huntington's disease. In a preferred embodiment, an RNA silencing agent of the invention targets any of the allelic polymorphisms of Huntingtin designated P1-P43 and listed in Tables 2 and 3 of International Publication No. WO 05/027980 or any of the Hungtintin SNP's (e.g., RS262125 or RS362331) described in U.S. Ser. No. 60/819,704 filed Jul. 6, 2006, both of which are incorporated by reference herein.

VI. Methods of Introducing Nucleic Acids, Vectors, and Host Cells

RNA silencing agents of the invention may be directly introduced into the cell (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the invention can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of RNA silencing agent material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of RNA silencing agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA silencing agent may be introduced in an amount which allows delivery of at least one copy per cell. Higher doses (e.g., at least 5, 10, 100, 500 or 1000 copies per cell) of material may yield more effective inhibition; lower doses may also be useful for specific applications.

VII. Pharmaceutical Compositions and Methods of Administration

The compounds (i.e., RNA silencing agents) of the invention can be incorporated into pharmaceutical compositions. Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

The compounds can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a nucleic acid molecule (i.e., an effective dosage) depends on the nucleic acid selected. For instance, if a plasmid encoding shRNA is selected, single dose amounts in the range of approximately 1 :g to 1000 mg may be administered; in some embodiments, 10, 30, 100 or 1000 :g may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The nucleic acid molecules of the invention can be inserted into expression constructs, e.g., viral vectors, retroviral vectors, expression cassettes, or plasmid viral vectors, e.g., using methods known in the art, including but not limited to those described in Xia et al., (2002), supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). supra; Miyagishi and Taira (2002), Nature Biotechnol., 20, 497-500; Paddison et al. (2002), supra; Paul (2002), supra; Sui (2002) supra; Yu et al. (2002), supra.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, Tuschl (2002), supra.

The pharmaceutical compositions can be included in a container, pack, or dispenser together with instructions for administration.

VIII. Methods of Treatment

The present invention provides for both prophylactic and therapeutic methods of treating a subject at risk of (or susceptible to) a disease or disorder caused, in whole or in part, by a gain-of-function mutant protein. In one embodiment, the disease or disorder is a dominant gain-or-function disease. In a preferred embodiment, the disease or disorder is a disorder associated with the an alteration of SOD 1 gene, specifically a point mutation in the SOD1 mutant allele, leading to a defect in SOD 1 gene (structure or function) or SOD1 protein (structure or function or expression), such that clinical manifestations include those seen in ALS disease patients.

“Treatment”, or “treating” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., a RNA silencing agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the invention pertains to methods treating subjects therapeutically, i.e., alter onset of symptoms of the disease or disorder. In an exemplary embodiment, the modulatory method of the invention involves contacting a cell expressing a gain-of-function mutant with a therapeutic agent (e.g., a RNA silencing agent or vector or transgene encoding same) that is specific for a mutation within the gene, such that sequence specific interference with the gene is achieved. These methods can be performed in vitro (e.g., by culturing the cell with the agent) or, alternatively, in vivo (e.g., by administering the agent to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in an appropriate animal model. For example, an RNA silencing agent (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

### EXAMPLES

The following materials, methods, and examples are illustrative only and not intended to be limiting.

General Methods

Preparation of Drosophila embryo lysate and target RNAs, cap labeling, siRNA annealing, and in vitro RNAi reactions were as described (Zamore et al., Cell, (2000), 101: 25-33; Haley et al., Methods, (2003), 30: 330-336; Tuschl et al., Genes Dev., (1999), 13:3191-3197). SOD1 mutant and wild-type RNAs were transcribed from BamHI-linearized plasmids (Crow et al., J. Neurochem, (1997), 69: 1936-1944) with recombinant histidine-tagged T7 RNA polymerase. Target RNAs and siRNAs were used at ˜5 nM and 50 nM final concentrations, respectively or ˜0.5 nM and 100 nM, respectively, for single turnover conditions. Gels were dried and exposed to phosphorimager plates (Fuji) and analyzed using a FLA-5000 phosphorimager (Fuji). Data was analyzed and quantified using ImageGuage 3.45 (Fuji), Excel X (Microsoft, Redmond, Wash.) and Igor Pro 5.01 (Wavemetrics, Lake Oswego, Oreg.).

Cell Culture, Transfection, and Luciferase Assays

HeLa cells were propagated and maintained as described (Schwarz et al, Mol Cell, (2002), 10: 537-548). HEK 293 cells were maintained in Dulbecco's Modified Eagle Media (DMEM) (Invitrogen), supplemented with 10% Fetal Bovine Serum, 100 U/ml penicillin, and 100 □g/ml streptomycin. HTT sequences were engineered into the 3′ UTR of the pRLTK Renilla luciferase vector (Promega, Madison, Wis.) using 55-bp DNA oligonucleotides (IDT, Coralville, Iowa) designed to create 5′ overhands when annealed in 1× lysis buffer (100 mM potassium acetate, 30 mM Hepes-KOH, pH 7.4, 2 mM magnesium acetate), allowing their insertion into the plasmid Xbal site. Plasmid constructs were verified by bidirectional sequencing. SOD1 sequences were cloned into the 3′ UTR of the firefly luciferase mRNA (pGL2 control, Promega) into NdeI and SpeI sites engineered into the plasmid by annealing two 39-nuceotide DNA oligos and ligating them into the vector. Transfections were carried out using LipofectAMINE 2000 (Invitrogen) in 24 well plates using 0.25 □g of pGL2 firefly luciferase (Promega) and 0.1 □g Renilla-HTT constructs or in 96 well plates using 2 □g/ml firefly fusion vector and 0.1 □g/ml Renilla vector. Cells were washed in 1× PBS (Invitrogen) and harvested 24 h after transfection in 1× passive lysis buffer (Promega). Luciferase levels were determined using the Dual Luciferase kit (Promega) and a Veritas Microplate Luminometer (Turner Biosystems, Sunnyvale, Calif.). Renilla luciferase/firefly luciferase ratios were normalized to a transfection with GFP siRNA (Qiagen, Valencia, Calif.). IC50 values were determined by fitting the data to the Hill Equation with n=1. For siRNAs in which the half maximal concentration for silencing was not reached at the highest concentration tested, IC 50 values were reported as greater than the highest concentration tested.

Microarray and Quantitative PCR Analysis

HeLa cells were from the American Type Culture Collection (Rockville, Md.). Cells were plated 24 hours prior to transfection with OligofectAMINE (Invitrogen). Duplexes were used at a final concentration of 100 nM. Cells were transfected in 6-well plates and RNA was isolated 24 hours following transfection. Total RNA was purified using the RNeasy kit (Qiagen). Microarray analysis was performed as described previously (Jackson A L et al., Nat. Biotechnol., (2003), 21: 635-637; Hughes T R et al., Nat. Biotechnol., (2001), 19: 342-347; Jackson A L et al., Nat. Biotechnol., (2001), 19: 342-347). Amplified cRNA from siRNA-transfected cells was hybridized against cRNA from mock-transfected cells (treated with transfection reagent in the absence of RNA duplex). Ratio hybridizations were performed with fluorescent label reversal to eliminate dye bias. Error models have been described previously (Jackson A L et al., Nat. Biotechnol., (2003), 21: 635-637; Hughes T R et al., Nat. Biotechnol., (2001), 19: 342-347; Jackson A L et al., Nat. Biotechnol., (2001), 19: 342-347). Data were analyzed using Rosetta Resolver (Rosetta Biosoftware, Seattle, Wash.). mRNA levels were also measured by quantitative RT-PCR using an ABI PRISM 7900HT Sequence Detection System and Assays-on-Demand gene expression products (Applied Biosystems [ABI], Foster City, Calif.). SOD1 mRNA was measured using ABI assay No. Hs00533490_A1 and normalized to β glucuronidase mRNA, measured using ABI assay No. 4310888E. Unprocessed microarray data has been deposited in the Gene Expression Omnibus database under the accession number GSE5291.

### Example I

### RNA Silencing of a Tiled Set of Functionally Asymmetric siRNAs Targeting Mutant SOD1

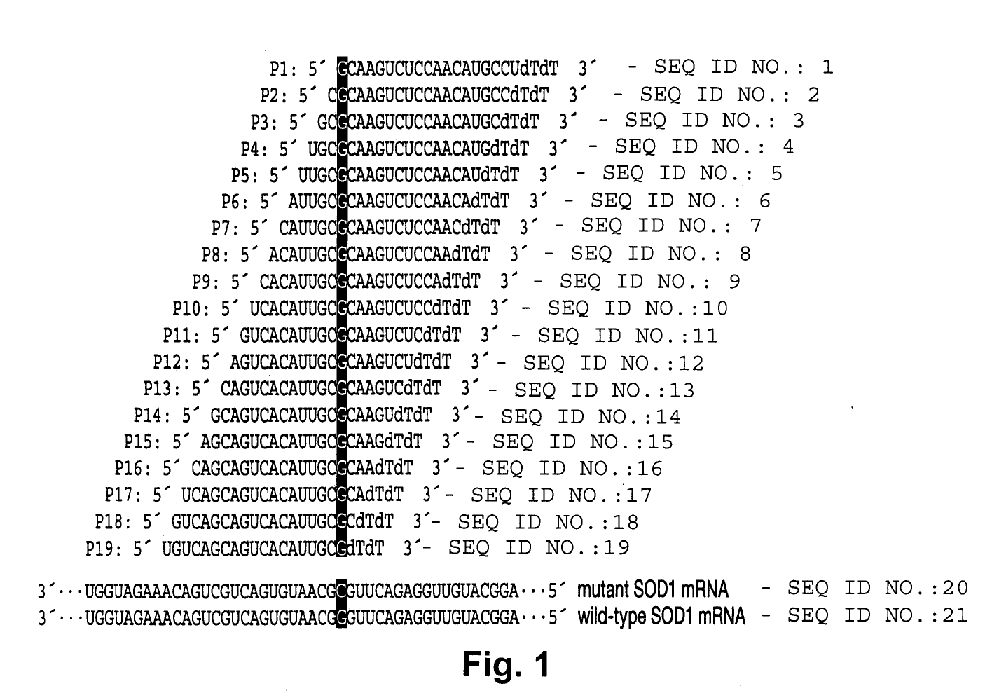

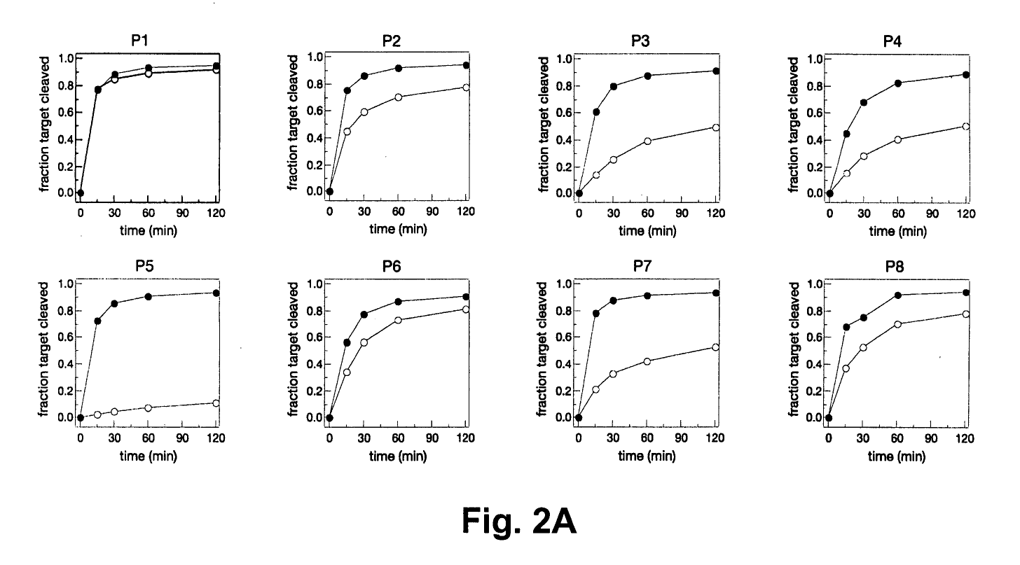

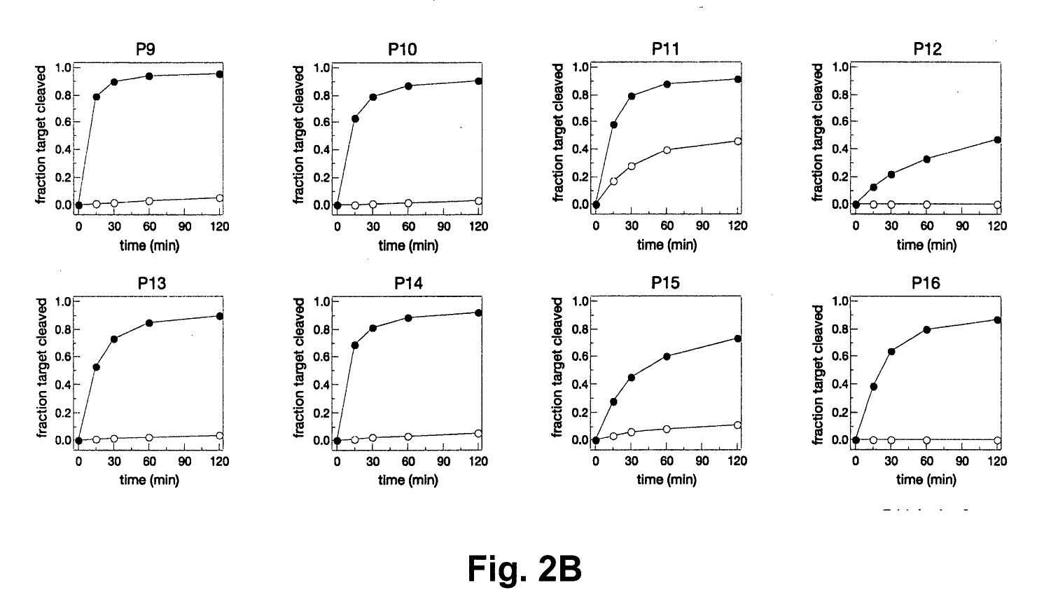

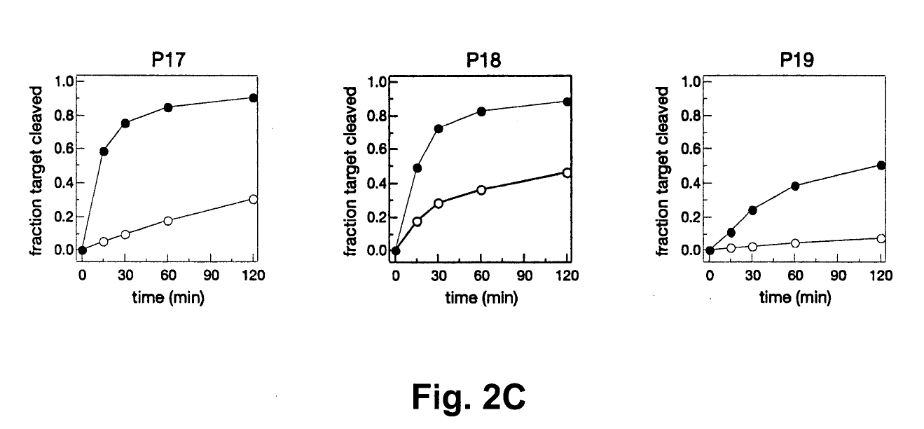

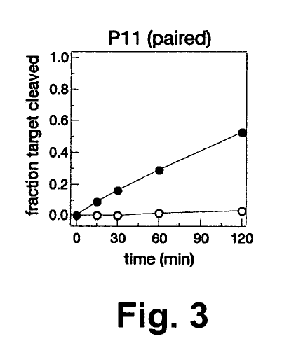

A set of 19 siRNAs (SEQ ID NOs 1-19) were designed by tiling across the G85R point mutation of human SOD1 (see FIG. 1). The G85R mutant (SEQ ID NO: 20) contains a cytosine at position 323 of the mRNA, whereas the wild-type mRNA (SEQ ID NO: 21) bears a guanosine at that position. Each siRNA fully matched the mutant SOD1, but contained a G:G mismatch with wild-type. To ensure that the antisense strand of each siRNA served as the guide in RISC, each siRNA had an unpaired, antisense-strand 5′ end, a design strategy that imparts ‘functional asymmetry’ to an siRNA (Schwarz D S, et al., Cell, 115: 199-208 (2003)). In vitro RNAi experiments using a cell-free Drosophila embryo lysate system demonstrated that each of the 19 siRNAs effectively targeted its fully matched target, the mutant G85R allele of SOD1, allowing assessment of how well each siRNA discriminated against the wild-type SOD1 allele (see FIGS. 2A-C). The importance of this strategy can be seen by comparing a conventionally designed (i.e., fully paired) P11 siRNA (FIG. 3) with the functionally asymmetric version of the P11 siRNA (FIG. 2B). The conventionally designed P11 siRNA showed considerable discrimination against the wild-type SOD1 allele, while the functionally asymmetric P11 siRNA revealed that the source of this discrimination was the relatively poor activity of the original siRNA against the fully matched target, rather than a large difference in its activities against the two SOD1 alleles.

Analysis of the tiled set of functionally asymmetric siRNAs showed that the P5, 9, 10, 13, 14, 15, and 16 siRNAs all discriminated between G85R mutant and wild-type SOD1 (FIGS. 2A-C). Additionally, the P12 and P19 siRNAs displayed some discrimination against the mismatched wild-type target, but these two siRNAs did not show robust silencing of the perfectly matched mutant target in the cell-free RNAi reaction, consistent with the idea that an unpaired guide strand 5′ end is not the sole determinant of siRNA efficacy (Khovorova et al., Cell, (2003), 115: 209-216, 2003; Reynolds et al., Nat. Biotechnol., (2004), 22: 326-330).

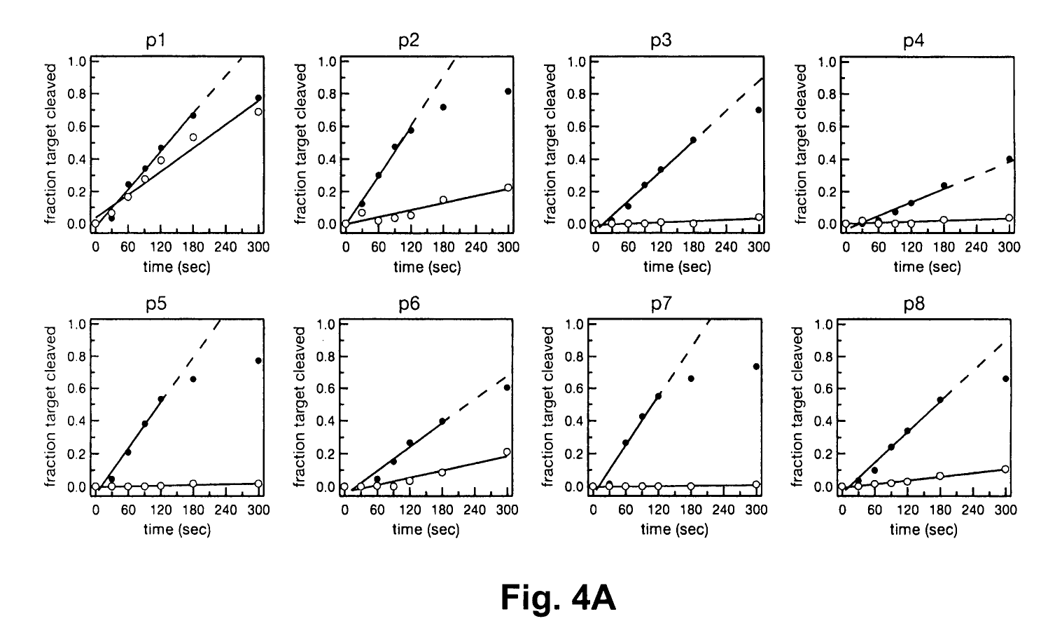

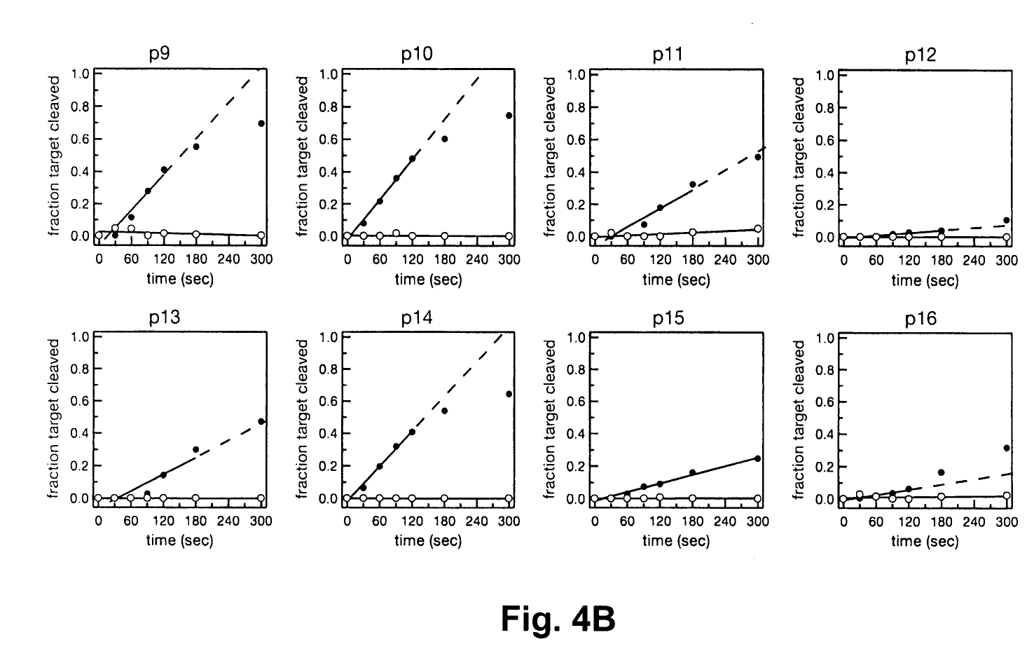

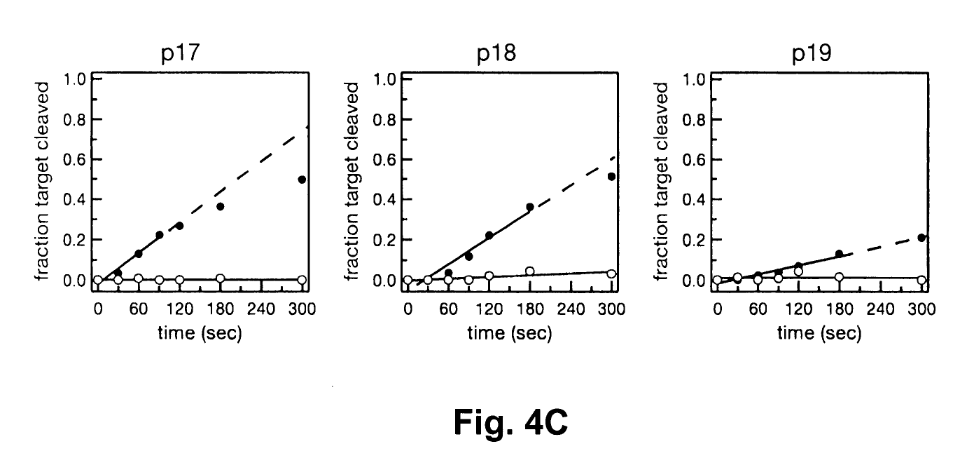

To provide a more quantitative measure of siRNA efficacy, we also determined for each siRNA in the tiled set its initial rate of cleavage in single-turnover reaction conditions (see FIGS. 4A and B). The initial rate of cleavage reflects the concentration of active RISC containing the antisense-strand of the siRNA duplex and the inherent catalytic rate of cleavage of the targeted sequence, but not the rate of product dissociation from RISC. Four siRNAs exhibited surprisingly slow initial rates of reaction: P12, P15, P16 and P19. Of these siRNAs, p12 and p19 also showed a low extent of cleavage over a longer time course (see FIGS. 2B and 2C). In contrast, the P15 and P16 siRNAs performed well over the two-hour time course, although they showed a slow rate of initial cleavage.

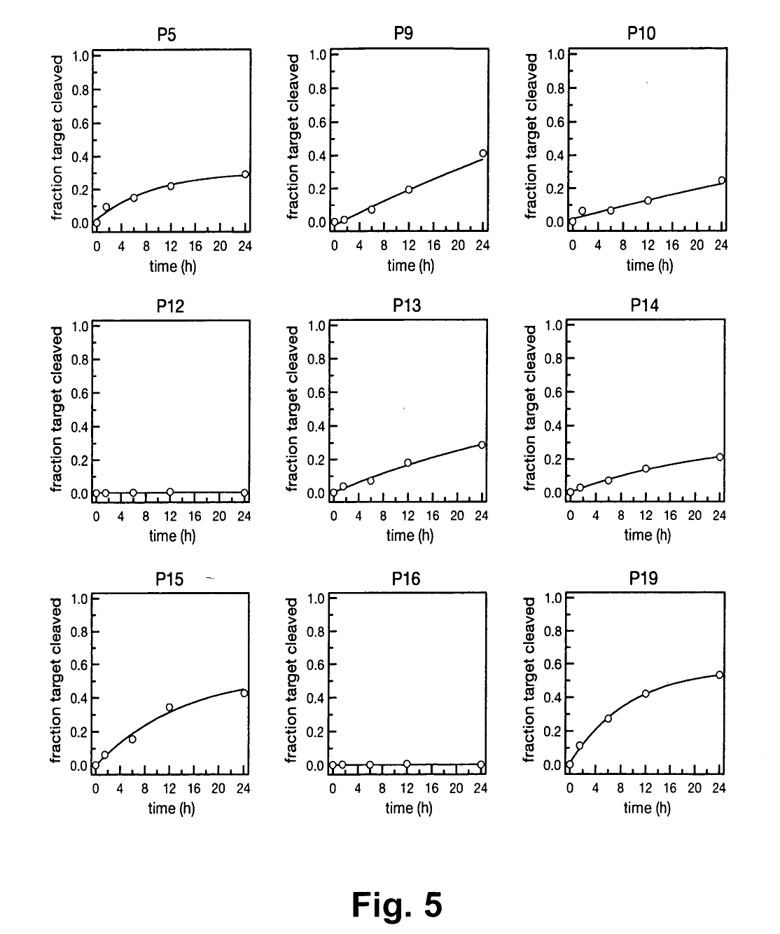

While some siRNAs exhibit high levels of discrimination during a 2 h reaction, a more rigorous test of the ability of an siRNA to discriminate against the mismatched RNA target is to examine cleavage over a 24 h period. Using a high concentration of siRNA and a low concentration of target RNA, 24 hour cleavage reactions were performed so as to detect even a small degree of activity of the siRNA against the mismatched target (see FIG. 5). Under these intentionally artificial conditions, many of the siRNAs which originally showed complete discrimination against the wild-type SOD1 RNA target, showed detectable levels of cleavage of the wild-type, mismatched RNA. In contrast, the P12 and P16 siRNAs, showed no cleavage of the wild-type target, suggesting that the purine:purine mismatch at these positions effectively blocked RISC activity under these experimental conditions.

### Example II

### Analysis of Tiled siRNAs in Cultured Human Cells

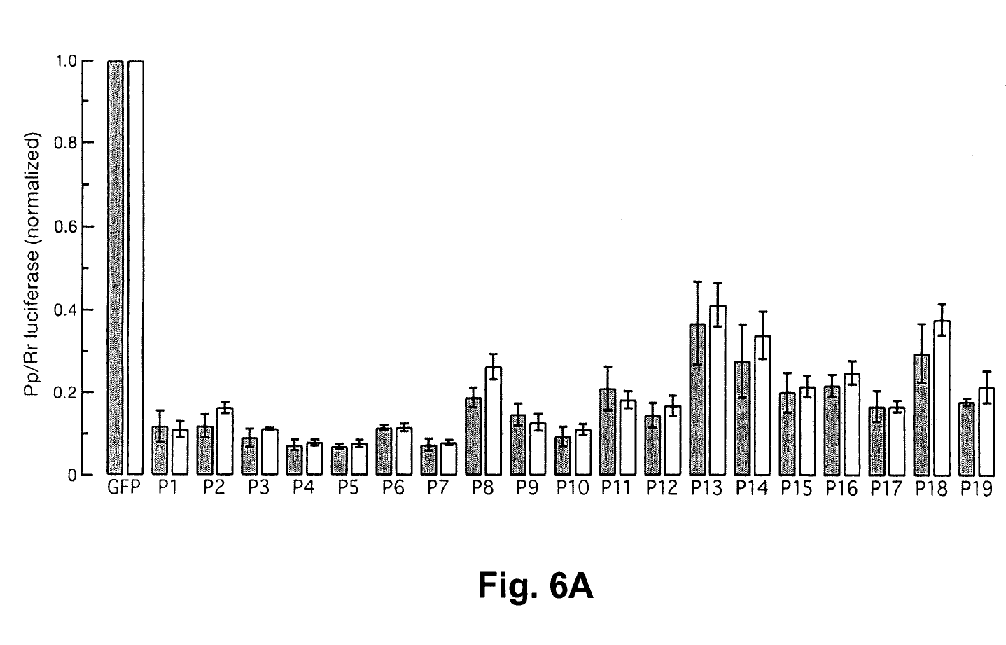

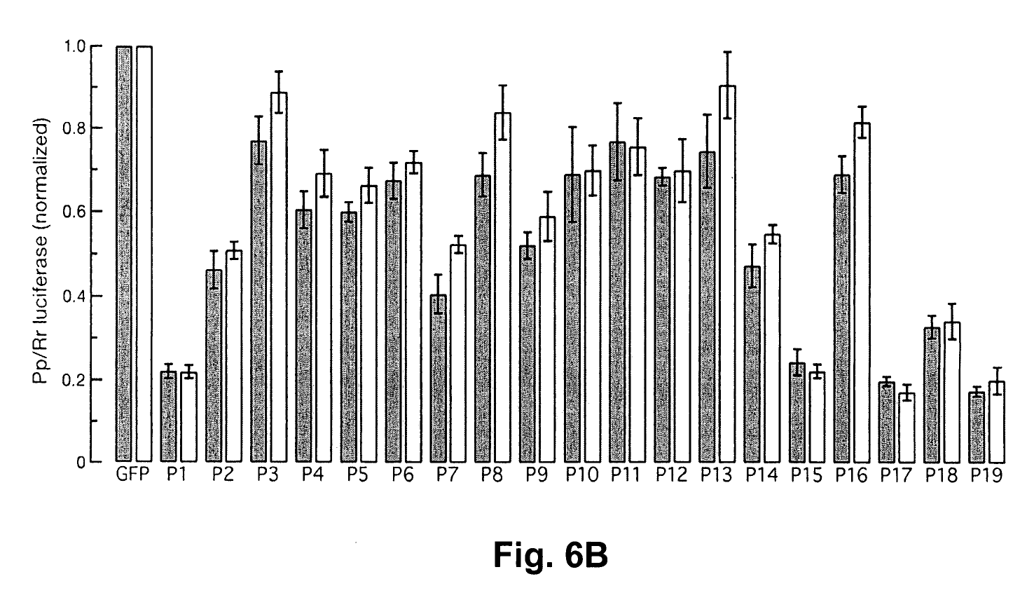

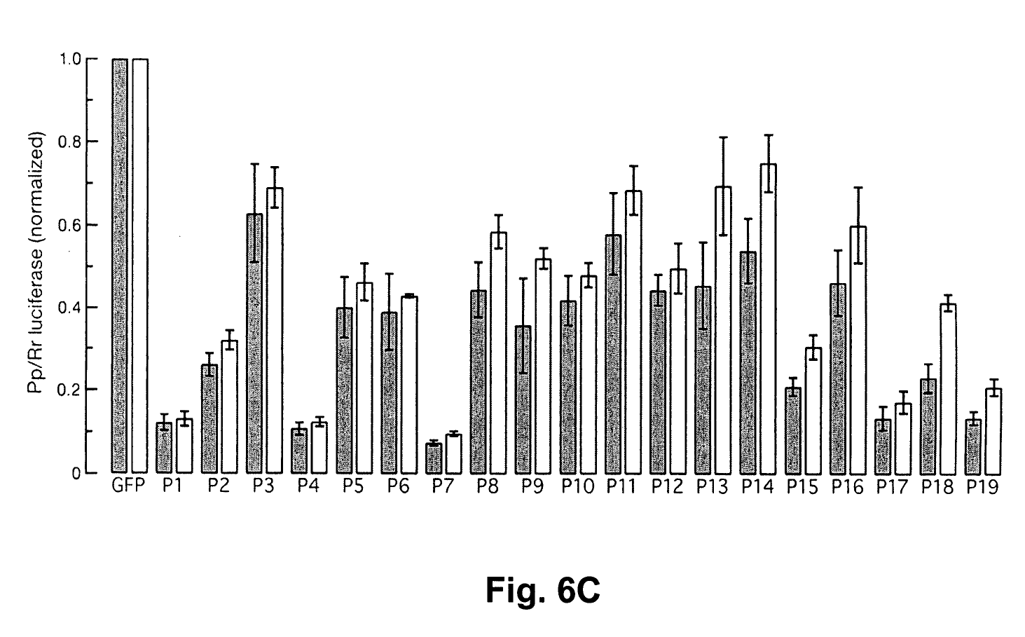

The efficacy and discriminatory power of each siRNA of the tiled set of siRNAs from Example I were examined in a human cell-based assay. SiRNAs were co-transfected into HEK 293 cells with a plasmid expressing a firefly (Photinus pyralis, Pp) luciferase bearing either the relevant region of the wild-type SOD1 or the G85R mutant sequence cloned into its 3′-untranslated region. Silencing efficiency was determined by measuring firefly luciferase activity, relative to an untargeted Renilla luciferase control, 24 h after transfection with either 2 nM or 20 nM siRNA (see FIGS. 6A-6C). Wild-type SOD1 contains a G at position 323; in the G85R mutant, this position is a C. The siRNAs were also evaluated using a Pp-luciferase-SOD1 fusion target containing a uridine residue at position 323 of the SOD1 mRNA sequence, thereby facilitating the formation of a G:U wobble base pair between the target mRNA and the “seed” region of the siRNA guide strand. The “seed” region of siRNA, which mediates siRNA binding to the target RNA, is thought to be highly sensitive to mismatches. Of the 19 siRNAs examined using the fully matched target RNA (the G85R mutant SOD1), all siRNAs silenced the reporter by at least 60%, with fifteen silencing the reporter by 80% or more (see FIG. 6A). When the same set was examined using the mismatched (i.e., G:G mismatched), wild-type SOD1 reporter, 10 of the 19 siRNAs effectively discriminated against the mismatched target RNA (FIG. 6B). siRNAs P3, P4, P5, P6, P8, P10, P11, P12, P13, and P16 all repressed wild-type reporter expression by less than 40%. Thus, most of the siRNAs that exhibited high levels of discrimination in the cell-free Drosophila RNAi system, also discriminated in cultured human cells, including siRNAs P5, P9, P10, P12, P13, P14, and P16.

Next, the same set of siRNAs was co-transfected with the reporter designed to create a G:U wobble instead of a G:G mismatch. Only five siRNAs of the 19 showed effective discrimination against the wild-type SOD1 reporter (i.e., less than a 40% reduction in expression)(FIG. 6C). Remarkably, siRNA P3 was the only one of the six siRNAs to show more than 2-fold allele specificity when a G:U wobble was placed within the seed sequence. Rather, siRNA with mismatches in the central siRNA:target RNA helix and 3′ to the seed (i.e. siRNAs P8, P11, P13, P14 and P16) best retained the ability to discriminate against the G:U wobble. Without being bound to any particular theory, it is hypothesized that seed mismatches are ineffective at destabilizing the binding of siRNAs bearing extensive complementarity to their targets, because base-pairs outside the seed region may compensate for mismatches within the seed (Brennecke J et al., PLoS Biol., (2005), 3: e85), whereas, mutations 3′ to the seed disrupt the A-form helical geometry required for target cleavage (Chiu Y L et al., Mol. Cell, (2002), 10: 549-561; Haley B et al., Nat. Struct. Mol. Biol., (2004), 11: 599-606).

### Example III

### Off-Target Effects of siRNAs Against Mutant SOD1

To examine the nature of off-target silencing (i.e., siRNA-dependent silencing of mRNAs unrelated to the intended target) triggered by each siRNA, asymmetric siRNAs were designed by unpairing the 5′ end of the guide strand to promote its incorporation into RISC. Because the siRNA seed sequence is the primary determinant of siRNA binding, off-target mRNAs contain six-nucleotide sequences (i.e., “hexamers”) complementary to the seed sequence of the siRNA strand (sense or antisense) that directed RISC to destroy them. Determining which strand gives the greatest enrichment of seed region hexamer matches to the off-target expression signatures is a measure of which siRNA strand is preferentially loaded into RISC.

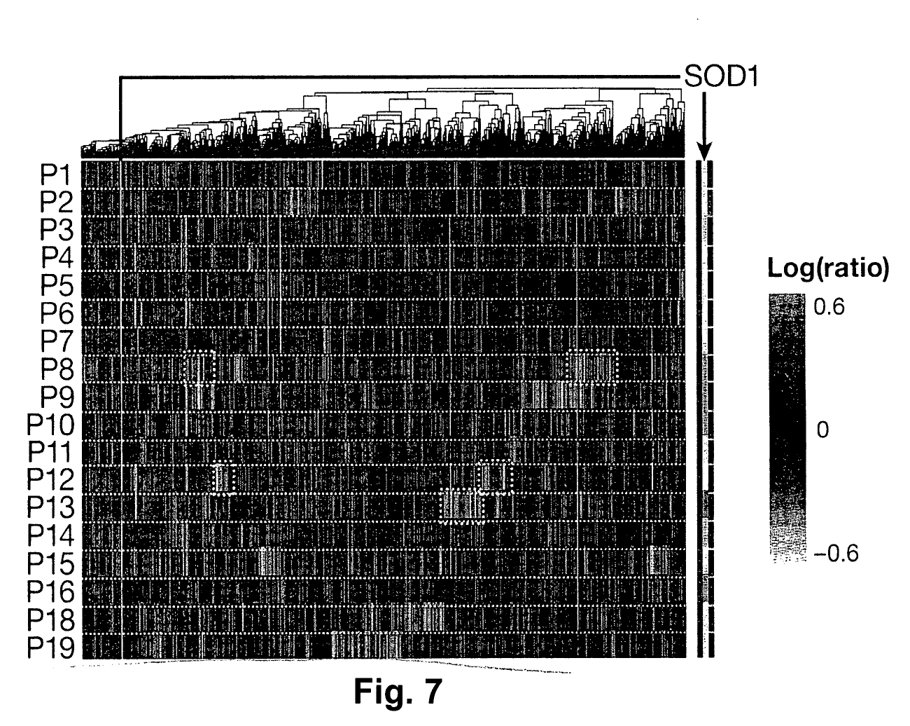

siRNAs were transfected into cultured human HeLa cells at 100 nM final concentration. This extraordinarily high siRNA concentration was selected to maximize off-target effects, in order to reveal the identity of the siRNA strand loaded into RISC. Total RNA was isolated from the siRNA transfected cells and analyzed by microarray transcription profiling (see FIG. 7 and Table 1). Each of the 19 siRNAs in the tiled set contains a different seed sequence, so each should have a characteristic off-target signature. In addition, this experiment poses a stringent test for siRNA specificity, in that (1) the transfected siRNA concentration was 5 times greater than the highest standard concentration (Semizarov et al., PNAS, (2003), 100: 6347-6352) and 50 times greater than the lower, effective concentration (2 nM) used in Example II; and (2) the endogenous, wild-type SOD1 mRNA is the human mRNA most complementary to each of the siRNAs that target mutant SOD1. (Human cell lines expressing the G85R allele of SOD1 are not available.)

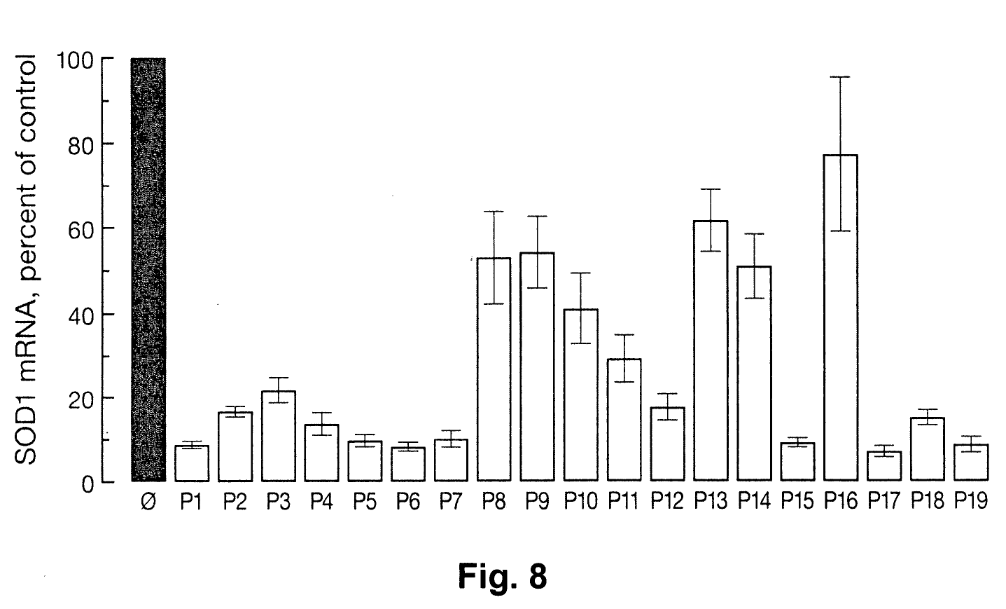

Analysis of the off-target genes down-regulated by the set of 18 siRNAs suggests that 8 loaded predominantly their antisense strand into RISC and 5 loaded both strands to some degree (see Table 1). siRNA strands were designated as active if the seed hexamer from that stand ranked in the top 20 hexamers (of 4,096 possible hexamers) and/or gave an E-value of enrichment of less than 0.003. The microarray data show that three siRNAs (P8, P9, and P16) triggered no detectable down-regulation of endogenous wild-type SOD1 (FIG. 7). Quantitative RT-PCR (qRT-PCR) corroborated the microarray analysis (see FIG. 8). The p16 siRNA detectably incorporated only the antisense strand into RISC, whereas both the P8 and P9 siRNA loaded both strands into RISC. The p9 and p16 siRNAs were also highly active in both Drosophila embryo lysate (see FIGS. 2B, 4B, and 5) and HEK 293T cells (see FIG. 6A) against the perfectly matched G85R mutant mRNA.

TABLE 1
Off-target Analysis of SOD1 siRNAs

| | | | | | | | Active |
|---|---|---|---|---|---|---|---|
| | hexamer | rank | E-value | hexamer | rank | E-value | Strand* |
| P1 | GACUUG | 1 | 3.06 × 10⁻⁸ | CAUGCC | 3 | 1.96 × 10⁻⁴ | Mixed |
| P2 | ACUUGC | 1 | 1.63 × 10⁻⁵⁵ | ACAUGC | >20 | 3.05 | AS |
| P3 | CUUGCG | 11 | 44.3 | CAACAU | >20 | 246 | AS |
| P4 | UUGCGC | 1 | 1.13 × 10⁻⁵ | CCAACA | >20 | 417 | AS |
| P5 | UGCGCA | >20 | 142 | CCAACA | 1 | 8.84 × 10⁻¹⁰ | S |
| P6 | GCGCAA | 20 | 1.64 | UCCAAC | 1 | 7.02 × 10⁻⁵ | S |
| P7 | CGCAAU | 1 | 1.08 × 10⁻⁵ | CUCCAA | >20 | 58.2 | AS |
| P8 | GCAAUG | 4 | 8.55 × 10⁻⁷ | GUCUCC | 1 | 1.65 × 10⁻¹⁷ | Mixed |
| P9 | CAAUGU | 1 | 5.24 × 10⁻⁴² | GUCUCC | 10 | 3.21 × 10⁻¹⁰ | Mixed |
| P10 | AAUGUG | 1 | 7.26 × 10⁻⁵ | AAGUCU | >20 | 317 | AS |
| P11 | AUGUGA | 1 | 2.13 × 10⁻⁵ | AAGUCU | >20 | 1.44 × 10³ | AS |
| P12 | UGUGAC | 3 | 4.31 × 10⁻¹⁴ | CAAGUC | 2 | 2.1 × 10⁻¹⁵ | Mixed |
| P13 | GUGACU | 1 | 2.92 × 10⁻²⁸ | GCAAGU | 2 | 4.63 × 10⁻¹⁹ | Mixed |
| P14 | UGACUG | >20 | 146 | GCGCAA | 1 | 5.68 × 10⁻² | S |
| P15 | GACUGC | 1 | 7.4 × 10⁻⁴⁷ | CGCAAG | >20 | 2.52 × 10³ | AS |
| P16 | GACUGC | 1 | 1.01 × 10⁻¹⁴ | UGCGCA | >20 | 599 | AS |
| P18 | UGCUGA | 1 | 3.3 × 10⁻⁵⁷ | CAUUGC | >20 | 5.71 | AS |
| P19 | GCUGAC | 1 | 1.12 × 10⁻⁶⁶ | ACAUUG | >20 | 5.06 × 10⁻³ | AS |

*AS = Antisense Strand; S = sense strand; Mixed = Both strands

Notably, all of the siRNAs bearing one G:G mismatch between the siRNA seed sequence and the endogenous wild-type SOD1 gene (P2, P3, P4, P5, P6, and P7) targeted the SOD1 mRNA for destruction at this high siRNA concentration (see FIGS. 7 and 8). That is, none of these siRNAs retained its ability to discriminate against wild-type SOD1 when the siRNA was transfected at 100 nM. These data are consistent with the view that mismatches between the seed and its target compromise only RISC binding, not catalysis, and can therefore be overcome by increasing the concentration of the siRNA (Haley B et al., Nat. Struct. Mol. Biol., (2004), 11: 599-606).

### Example IV

### Analysis of Additional Mismatches Using p10 siRNA

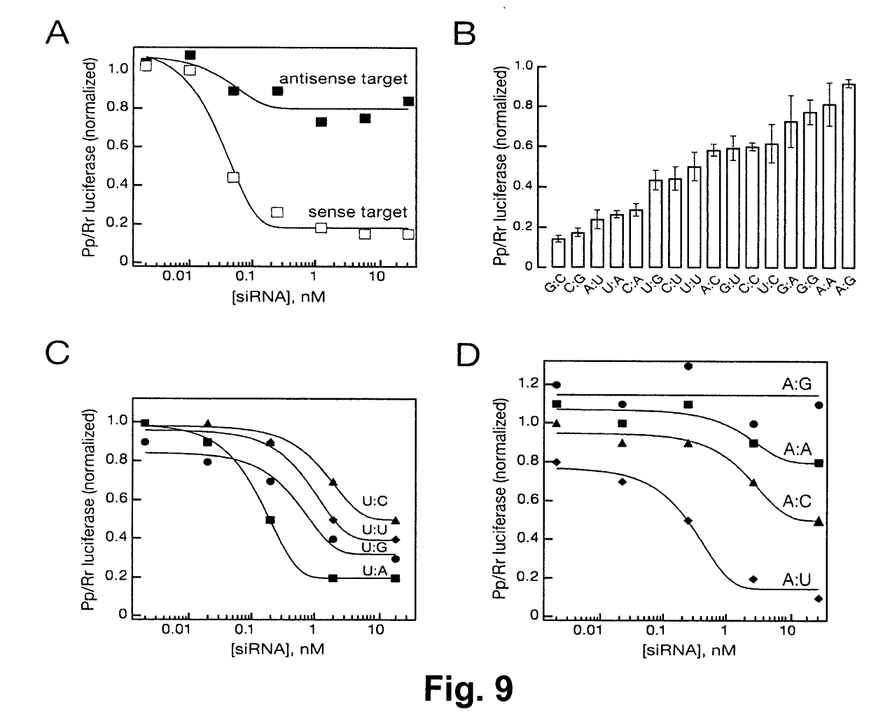

Examination of the tiled set of siRNAs targeting the G85R SOD1 mutation in the Examples supra compared a G:C base pair to a G:G mismatch and a G:U wobble. To extend this analysis to other types of mismatches, four siRNAs were synthesized based on the P10 siRNA sequence (SEQ ID NO: 10, FIG. 1), by placing a G, C, U, or A at position 10 of the siRNA and constructing four corresponding reporter constructs expressing Pp-luciferase targets RNAs containing each possible nucleotide across from siRNA position 10. Combining these four siRNAs with the four reporter constructs allowed examination of all possible position 10 matches and mismatches between the siRNA and the target. The siRNA sequence used in these studies was intrinsically asymmetric, silencing a reporter complementary to the siRNA antisense strand greater than 8-fold more effectively than a reporter complementary to the siRNA sense strand (see FIG. 9A).

Analysis of all possible siRNA:target pairs using 2 nM siRNA concentration revealed that the strength of pairing and compatibility with an A-form RNA:RNA helix between the siRNA and its target at siRNA position 10 correlated with silencing efficacy. Notably, at least one full A-form helical turn is required for an siRNA to direct cleavage of its RNA target (Chiu Y L et al., Mol. Cell, (2002), 10: 549-561; Haley B et al., Nat. Struct. Mol. Biol., (2004), 11: 599-606). While all the perfectly matched siRNAs (G:C, C:G, A:U, and U:A) effectively silenced the reporter, G:C and C:G pairs were the most active. Mismatches expected to be well accommodated in an A-form RNA:RNA helix (pyrimidine:pyrimidine, pyrimidine:purine, or purine:pyrimidine) displayed intermediate levels of discrimination, whereas purine:purine mismatches, expected to destabilize the helix or to promote a stable, non-helical, conformation, silenced the reporter least (see FIG. 9B). Increasing the siRNA concentration increased the extent of silencing (i.e., decreased single-nucleotide discrimination) for all siRNA:target combinations, except for the A:G mismatch, which maintained its ability to discriminate against the mismatched reporter at 20 nM siRNA (see FIGS. 9C and 9D).

### Example V

### Analysis of Purine:Purine Mismatches Across the siRNA Sequence

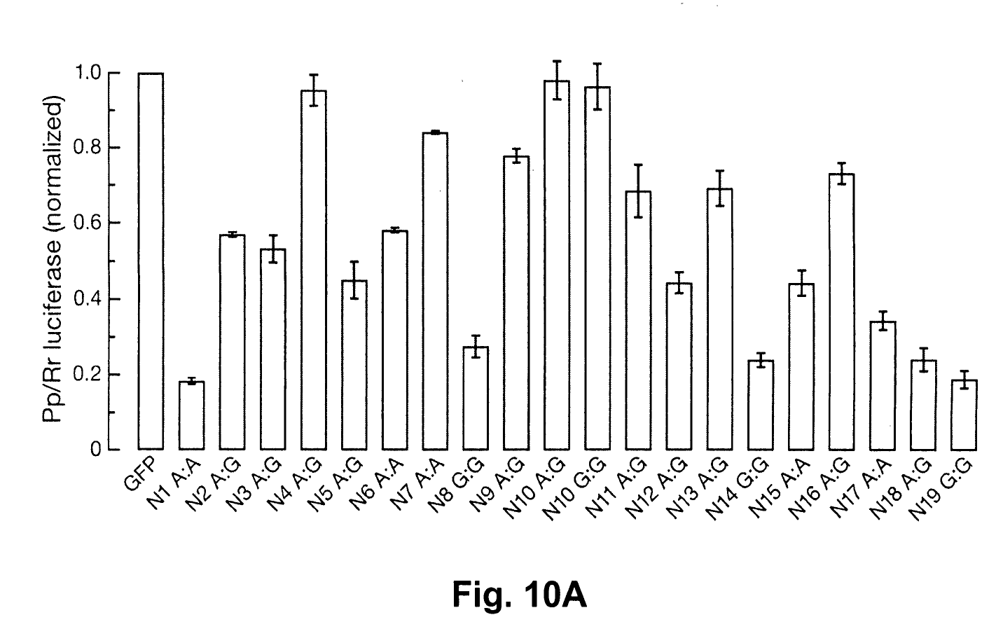

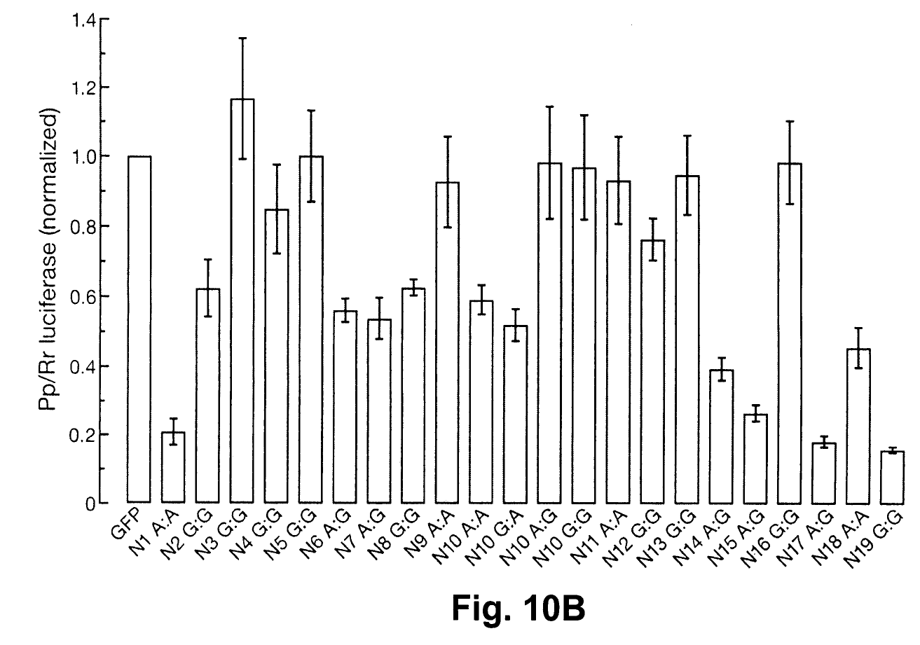

The above Examples suggest that purine:purine mismatches provide the highest level of discrimination against mismatched targets. To corroborate these findings, the effect of a purine:purine mismatch at the 19 positions (N1-N19) was examined in a single siRNA sequence: the P10 siRNA. For each purine position in the P10 siRNA, a reporter was constructed that expressed a Pp luciferase mRNA with a purine at the corresponding target position. For pyrimidine positions in the P10 siRNA, a variant siRNA was synthesized substituting a single pyrimidine with a purine so as to create a purine:purine clash with the reporter mRNA. Seven siRNAs reduced expression of the mismatched reporter to less than 40% of the unsilenced level: N4, N7, N9, N10, N11, N13 and N16 (FIG. 10A).

The same method of analysis was applied to the P4 siRNA (see FIG. 10B). Luciferase silencing was disrupted the least by siRNA:target mRNA combinations that placed a single purine:purine mismatch at siRNA guide positions 3, 4, 5, 9, 10, 11, 12, 13, or 16. Intriguingly, seed sequence mismatches—at positions 3, 4, and 5—were strongly discriminatory for this siRNA, which has the most thermodynamically stable seed sequence pairing of all the siRNAs in this study.

For the P4 siRNA scaffold, G:G and A:G mismatches at position 10 were more selective than A:A or G:A mismatches. The effect of such mismatches flanked by U:A base pairs has not been experimentally determined, but single-nucleotide base pairs can either stabilize or destabilize a helix, depending on the identity of both the mismatch and the adjacent base pairs (Kierzek, et al. Biochemistry, (1999), 38: 14214-14223).

### Example VI

### Position 16 Mismatches Discriminate Well

Throughout the above analyses (Examples I-V)—including cell-free RNAi reactions, reporter transfections, and microarray and quantitative-PCR analysis of endogenous mRNA—purine:purine mismatches at siRNA position 16 consistently discriminated against the mismatched target. Therefore, the generality of an siRNA position 16 mismatch as a strategy for designing allele-specific siRNA, was examined by synthesizing 10 distinct siRNA-mRNA pairs bearing G:G, A:G, or A:A mismatches (five targeting a SOD1 point mutation (SEQ ID NOs 22-26) and five targeting an HTT SNP (SEQ ID NOs 27-31)). For comparison, a fully matched siRNA was synthesized for each target. Each pair of mismatched and matched siRNAs targeted a site inserted into the 3′ untranslated region of Renilla or firefly luciferase (see Table 2). Reporter silencing, relative to a cotransfected firefly luciferase control, was determined for each siRNA over a concentration range from 0.001 nM to 20 nM. For each siRNA, the siRNA concentration producing half-maximal silencing (IC50) was calculated for the match or mismatched siRNAs (Table 2).

For all of the ten siRNA pairs tested, the IC50 for the siRNA:target combination with the position 16 mismatch was greater than for the fully matched siRNAs. For seven of the ten siRNA pairs, the IC50 was at least 20-fold greater for mismatched siRNA:target combination. Similar discrimination was observed in both HeLa and 293 cells (data not shown).

TABLE 2
Effect of Mismatches at Position 16

| | | | | |
|---|---|---|---|---|
| SEQ | | Match | Mismatch | |
| ID | siRNA Guide Strand | IC50 | IC50 | |
| NO: | (Position 16 mismatch) | (nM) | (nM) | Discrimination |

| | | | | |
|---|---|---|---|---|
| 22 | 5′ UCACAUUGCCCAAGUAUCCdTdT 3′ | 1.0 | >20 | >20 |
| 23 | 5′ UGCCCAAGUCUCCAAGAUGdTdT 3′ | 0.2 | >20 | >100 |
| 24 | 5′ CAGCAGUCACAUUGCGCAAdTdT 3′ | 0.9 | >20 | >22 |
| 25 | 5′ AGUCACAUUGCCCAAGUCUdTdT 3′ | 0.4 | >20 | >50 |
| 26 | 5′ CCAAGUCUCCAACAUGCCUdTdT 3′ | 0.9 | >20 | >22 |
| 27 | 5′ UGAAGUGCACACAGUGGAUGA 3′ | 0.17 | 0.73 | 4.3 |
| 28 | 5′ UGAAGUGCACACAGUAGAUGA 3′ | 0.1 | 0.43 | 4.3 |
| 29 | 5′ GAUGAAGUGCACACAGUGGAU 3′ | 0.15 | 20 | 133 |
| 30 | 5′ GUGCACACAGUGGAUGAGGGA 3′ | 0.23 | 2 | 8.6 |
| 31 | 5′ AGGGUCAAGAUGACAAUGGAC 3′ | 0.7 | >20 | >28 |

### Example VII

### Inosine-Modified siRNAs with Enhanced Discriminatory RNA Silencing Activity

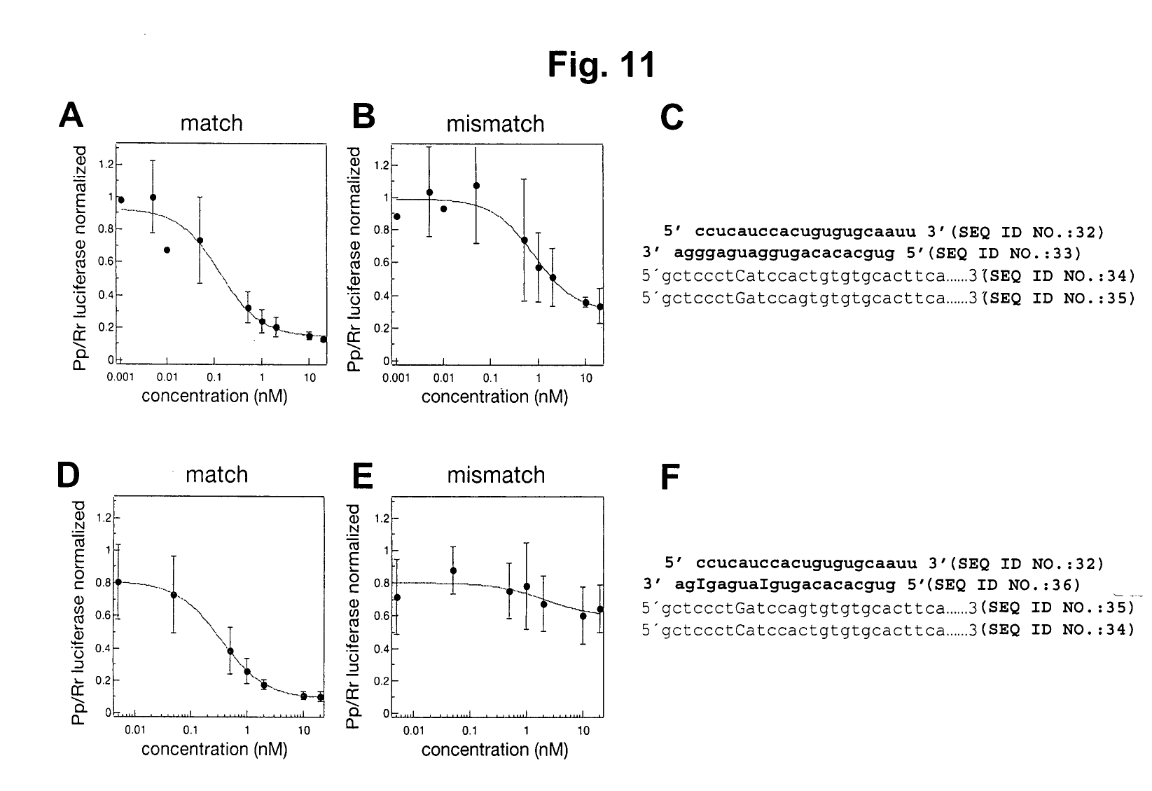

To assess quantitatively if an Inosine-modified siRNA duplex has enhanced discriminatory RNA silencing activity, the RNAi activity of an Inosine-modified siRNA was compared with that of an unmodified siRNA in a dual-luciferase reporter gene assay. Briefly, synthetic Pp luciferase reporter mRNAs were constructed containing the relevant portion of a target mutant huntingtin mRNA sequence (SEQ ID NO:34) or a non-target, wild-type huntingtin mRNA sequence (SEQ ID NO:35) (see FIG. 11C). An unmodified siRNA duplex was designed having an antisense strand (SEQ ID NO:33) perfectly complementary (“matched”) to the target mRNA sequence, while having a G:G mismatch with the non-target mRNA sequence. A corresponding Inosine-modified siRNA was also synthesized having two inosine residues (“I”) in the antisense strand. As shown in FIGS. 11A and 11B, the unmodified siRNA duplex potently inhibited Pp luciferase expression relative to an internal Rr luciferase control (IC50˜0.1 nM, see FIG. 11A), while also significantly inhibiting expression of the mismatched, non-target mRNA sequence (IC50˜2-3 nM). In contrast, as shown in FIGS. 11D and 11E the Inosine-modified siRNA exhibited virtually no silencing of the mismatched non-target mRNA (see FIG. 11E), while retaining virtually identical silencing activity against the target mRNA sequence (see FIG. 11D). Accordingly, these results indicate that siRNAs modified with destabilizing nucleotides are surprisingly improved discriminatory RNA silencing properties.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

In addition, the contents of all patent publications discussed supra are incorporated in their entirety by this reference.

What is claimed is:

1. A method of enhancing discriminatory RNA silencing by an RNA silencing agent comprising positioning a specificity-determining nucleotide of said agent at a nucleotide position within an antisense strand of said agent, wherein the nucleotide position is 3′ of the seed sequence of said antisense strand, such that discriminatory RNA silencing is enhanced.

2. The method of claim 1, wherein the specificity-determining nucleotide forms a mismatched or wobble base pair with a non-target mRNA.

3. The method of claim 2, wherein the mismatched base-pair is a purine:purine mismatch.

4. The method of claim 3, wherein the purine mismatch is a G:G mismatch.

5. The method of claim 1, wherein the specificity-determining nucleotide forms a Watson-Crick base pair with a target mRNA.

6. The method of claim 1 or 4, wherein the nucleotide position is selected from the group consisting of P9, P10, P12, P13, P14, P15, P16 and P19, and wherein said nucleotide position is relative to the 5′end of the antisense strand.

7. The method of claim 6, wherein the nucleotide position is P10.

8. The method of claim 6, wherein the nucleotide position is P16.

9. The method of claim 1, wherein discriminatory RNA silencing by the RNA silencing agent is further enhanced by substituting at least one nucleotide within the antisense strand with a destabilizing nucleotide.

10. The method of claim 9, wherein the destabilizing nucleotide is a universal base.

11. The method of claim 10, wherein the universal base is inosine or 2′-deoxyinosine.

12. The method of claim 9, wherein the destabilizing nucleotide is position within 5 nucleotide positions of the specificity-determining nucleotide.

13. The method of claim 2, wherein the non-target RNA is encoded by a wild-type allele corresponding to the mutant allele of a gene encoding a mutant gain-of-function protein.

14. The method of claim 5, wherein the specificity-determining nucleotide forms a Watson-Crick base pair with a single-nucleotide polymorphism associated with a mutant allele of a gene encoding a mutant gain-of-function protein.

15. The method of claim 13 or 14, wherein said mutant gain-of-function protein is a mutant SOD1 or Hungtingtin protein.

16. The method of claim 1, wherein the RNA silencing agent is a siRNA.

17. The method of claim 1, wherein the RNA silencing agent provides more than 4-fold discrimination between two alleles which differ by at least one nucleotide.

18. An RNA silencing agent synthesized according the method of claim 1.

19. A method of enhancing discriminatory RNA silencing by an RNA silencing agent comprising substituting at least one nucleotide within an antisense strand of said agent with a destabilizing nucleotide, such that discriminatory RNA silencing by said RNA silencing agent is enhanced.

20. The method of claim 19, wherein the antisense strand has a G/C content of greater than 40%.

21. The method of claim 19, wherein the RNA silencing agent is capable of inducing the discriminatory RNA silencing of a target sequence having a G/C content of greater than 40%.

22. The method of claim 19, wherein the melting temperature (Tm) of a duplex formed by said antisense strand and a corresponding target mRNA sequence is decreased.

23. The method of claim 22, wherein the Tm is decreased by more than 0.5° C.

24. The method of claim 22, wherein the Tm is decreased by less than 2° C.

25. The method of claim 19, wherein the destabilizing nucleotide is a universal base.

26. The method of claim 19, wherein the universal base is selected from the group consisting of inosine and 2′-deoxyinosine.

27. The method of claim 19, wherein the nucleotide with the antisense strand is a G or C.

28. The method of claim 19, wherein the destabilizing nucleotide forms a base pair with a C in the target sequence.

29. The method of claim 19, wherein discriminatory RNA silencing by the RNA silencing agent is further enhanced by positioning a specificity-determining nucleotide of said agent at a nucleotide position within the antisense strand of said agent, wherein the nucleotide position is 3′ of the seed sequence of said antisense strand.

30. The method of claim 29, wherein the nucleotide position is selected from the group consisting of P9, P10, P12, P13, P14, P15, P16 and P19, and wherein said nucleotide position is relative to the 5′end of the antisense strand.

31. The method of claim 30, wherein the nucleotide position is P10.

32. The method of claim 6, wherein the nucleotide position is P16.

33. The method of method of claim 19 or 29, wherein the destabilizing nucleotide is present at a position within 5 nucleotides of the specificity-determining nucleotide.

34. The method of claim 19, wherein the RNA silencing agent is capable of substantially silencing a mutant allele of a gain-of-function protein without substantially silencing a corresponding wild-type allele.

35. The method of claim 19, wherein a specificity-determining nucleotide of the RNA silencing agent forms a Watson-Crick base pair with a single-nucleotide polymorphism associated with a mutant allele of a gene encoding a mutant gain-of-function protein.

36. The method of claim 34 or 35, wherein said mutant gain-of-function protein is a mutant SOD1 or Hungtingtin protein.

37. The method of claim 19, wherein the RNA silencing agent provides more than 4-fold discrimination between two alleles which differ by at least one nucleotide.

38. An RNA silencing agent synthesized according the method of claim 19.

39. A method of treating a subject having a disease or disorder correlated with the presence of a dominant gain of function mutant allele, the method comprising administering to the subject a therapeutically effective amount of an RNA silencing agent synthesized according to the method of claim 1 or 19.

40. The method of claim 39, wherein the disease is a neurodegenerative disease.

41. The method of claim 40, wherein the neurodegenerative disease is selected from the group of amyotrophic lateral sclerosis, Huntington's disease, Alzheimer's disease, Parkinson's disease, and spinocerebellar ataxia (SCA).

42. A method of enhancing discriminatory RNA silencing by an RNA silencing agent comprising positioning a specificity-determining nucleotide of said agent at nucleotide position P10 or P 16 relative to the 5′ end of an antisense strand of said agent, wherein (i) the nucleotide position is 3′ of the seed sequence of said antisense strand, (ii) the specificity-determining nucleotide forms a purine:purine mismatch with a non-target mRNA; and (iii) the specificity-determining nucleotide forms a Watson-Crick base pair with a target mRNA, such that discriminatory RNA silencing is enhanced.


SEQUENCE LISTING (36 sequences)

SEQ ID NO: 1 - DNA, 21 bp, Artificial Sequence
  Description of Combined DNA/RNA Molecule Synthetic oligonucleotide
  Description of Artificial Sequence Synthetic oligonucleotide
gcaagucuccaacaugccutt

SEQ ID NO: 2 - DNA, 21 bp, Artificial Sequence
  Description of Combined DNA/RNA Molecule Synthetic oligonucleotide
  Description of Artificial Sequence Synthetic oligonucleotide
cgcaagucuccaacaugcctt

SEQ ID NO: 3 - DNA, 21 bp, Artificial Sequence
  Description of Combined DNA/RNA Molecule Synthetic oligonucleotide
  Description of Artificial Sequence Synthetic oligonucleotide
gcgcaagucuccaacaugctt

SEQ ID NO: 4 - DNA, 21 bp, Artificial Sequence
  Description of Combined DNA/RNA Molecule Synthetic oligonucleotide
  Description of Artificial Sequence Synthetic oligonucleotide
ugcgcaagucuccaacaugtt

SEQ ID NO: 5 - DNA, 21 bp, Artificial Sequence
  Description of Combined DNA/RNA Molecule Synthetic oligonucleotide
  Description of Artificial Sequence Synthetic oligonucleotide
uugcgcaagucuccaacautt

SEQ ID NO: 6 - DNA, 21 bp, Artificial Sequence
  Description of Combined DNA/RNA Molecule Synthetic oligonucleotide
  Description of Artificial Sequence Synthetic oligonucleotide
auugcgcaagucuccaacatt

SEQ ID NO: 7 - DNA, 21 bp, Artificial Sequence
  Description of Combined DNA/RNA Molecule Synthetic oligonucleotide
  Description of Artificial Sequence Synthetic oligonucleotide
cauugcgcaagucuccaactt

SEQ ID NO: 8 - DNA, 21 bp, Artificial Sequence
  Description of Combined DNA/RNA Molecule Synthetic oligonucleotide
  Description of Artificial Sequence Synthetic oligonucleotide
acauugcgcaagucuccaatt

SEQ ID NO: 9 - DNA, 21 bp, Artificial Sequence
  Description of Combined DNA/RNA Molecule Synthetic oligonucleotide
  Description of Artificial Sequence Synthetic oligonucleotide
cacauugcgcaagucuccatt

SEQ ID NO: 10 - DNA, 21 bp, Artificial Sequence
  Description of Combined DNA/RNA Molecule Synthetic oligonucleotide
  Description of Artificial Sequence Synthetic oligonucleotide
ucacauugcgcaagucucctt

SEQ ID NO: 11 - DNA, 21 bp, Artificial Sequence
  Description of Combined DNA/RNA Molecule Synthetic oligonucleotide
  Description of Artificial Sequence Synthetic oligonucleotide
gucacauugcgcaagucuctt

SEQ ID NO: 12 - DNA, 21 bp, Artificial Sequence
  Description of Combined DNA/RNA Molecule Synthetic oligonucleotide
  Description of Artificial Sequence Synthetic oligonucleotide
agucacauugcgcaagucutt

SEQ ID NO: 13 - DNA, 21 bp, Artificial Sequence
  Description of Combined DNA/RNA Molecule Synthetic oligonucleotide
  Description of Artificial Sequence Synthetic oligonucleotide
cagucacauugcgcaaguctt

SEQ ID NO: 14 - DNA, 21 bp, Artificial Sequence
  Description of Combined DNA/RNA Molecule Synthetic oligonucleotide
  Description of Artificial Sequence Synthetic oligonucleotide
gcagucacauugcgcaagutt

SEQ ID NO: 15 - DNA, 21 bp, Artificial Sequence
  Description of Combined DNA/RNA Molecule Synthetic oligonucleotide
  Description of Artificial Sequence Synthetic oligonucleotide
agcagucacauugcgcaagtt

SEQ ID NO: 16 - DNA, 21 bp, Artificial Sequence
  Description of Combined DNA/RNA Molecule Synthetic oligonucleotide
  Description of Artificial Sequence Synthetic oligonucleotide
cagcagucacauugcgcaatt

SEQ ID NO: 17 - DNA, 21 bp, Artificial Sequence
  Description of Combined DNA/RNA Molecule Synthetic oligonucleotide
  Description of Artificial Sequence Synthetic oligonucleotide
ucagcagucacauugcgcatt

SEQ ID NO: 18 - DNA, 21 bp, Artificial Sequence
  Description of Combined DNA/RNA Molecule Synthetic oligonucleotide
  Description of Artificial Sequence Synthetic oligonucleotide
gucagcagucacauugcgctt

SEQ ID NO: 19 - DNA, 21 bp, Artificial Sequence
  Description of Combined DNA/RNA Molecule Synthetic oligonucleotide
  Description of Artificial Sequence Synthetic oligonucleotide
ugucagcagucacauugcgtt

SEQ ID NO: 20 - RNA, 45 nt, Artificial Sequence
  Description of Artificial Sequence Synthetic oligonucleotide
aggcauguuggagacuugcgcaaugugacugcugacaaagauggu

SEQ ID NO: 21 - RNA, 45 nt, Artificial Sequence
  Description of Artificial Sequence Synthetic oligonucleotide
aggcauguuggagacuugggcaaugugacugcugacaaagauggu

SEQ ID NO: 22 - DNA, 21 bp, Artificial Sequence
  Description of Combined DNA/RNA Molecule Synthetic oligonucleotide
  Description of Artificial Sequence Synthetic oligonucleotide
ucacauugcccaaguaucctt

SEQ ID NO: 23 - DNA, 21 bp, Artificial Sequence
  Description of Combined DNA/RNA Molecule Synthetic oligonucleotide
  Description of Artificial Sequence Synthetic oligonucleotide
ugcccaagucuccaagaugtt

SEQ ID NO: 24 - DNA, 21 bp, Artificial Sequence
  Description of Combined DNA/RNA Molecule Synthetic oligonucleotide
  Description of Artificial Sequence Synthetic oligonucleotide
cagcagucacauugcgcaatt

SEQ ID NO: 25 - DNA, 21 bp, Artificial Sequence
  Description of Combined DNA/RNA Molecule Synthetic oligonucleotide
  Description of Artificial Sequence Synthetic oligonucleotide
agucacauugcccaagucutt

SEQ ID NO: 26 - DNA, 21 bp, Artificial Sequence
  Description of Combined DNA/RNA Molecule Synthetic oligonucleotide
  Description of Artificial Sequence Synthetic oligonucleotide
ccaagucuccaacaugccutt

SEQ ID NO: 27 - RNA, 21 nt, Artificial Sequence
  Description of Artificial Sequence Synthetic oligonucleotide
ugaagugcacacaguggauga

SEQ ID NO: 28 - RNA, 21 nt, Artificial Sequence
  Description of Artificial Sequence Synthetic oligonucleotide
ugaagugcacacaguagauga

SEQ ID NO: 29 - RNA, 21 nt, Artificial Sequence
  Description of Artificial Sequence Synthetic oligonucleotide
gaugaagugcacacaguggau

SEQ ID NO: 30 - RNA, 21 nt, Artificial Sequence
  Description of Artificial Sequence Synthetic oligonucleotide
gugcacacaguggaugaggga

SEQ ID NO: 31 - RNA, 21 nt, Artificial Sequence
  Description of Artificial Sequence Synthetic oligonucleotide
agggucaagaugacaauggac

SEQ ID NO: 32 - RNA, 21 nt, Artificial Sequence
  Description of Artificial Sequence Synthetic oligonucleotide
ccucauccacugugugcaauu

SEQ ID NO: 33 - RNA, 21 nt, Artificial Sequence
  Description of Artificial Sequence Synthetic oligonucleotide
gugcacacaguggaugaggga

SEQ ID NO: 34 - DNA, 27 bp, Artificial Sequence
  Description of Artificial Sequence Synthetic oligonucleotide
gctccctcatccactgtgtgcacttca

SEQ ID NO: 35 - DNA, 27 bp, Artificial Sequence
  Description of Artificial Sequence Synthetic oligonucleotide
gctccctgatccagtgtgtgcacttca

SEQ ID NO: 36 - RNA, 21 nt, Artificial Sequence
  Description of Artificial Sequence Synthetic oligonucleotide
  modified_base (13): inosine
  modified_base (19): inosine
gugcacacagugnaugagnga